(19) United States
(12) Patent Application Publication
(10) Pub. No.: US 2024/0337647 A1
(43) Pub. Date: Oct. 10, 2024

(54) IMPROVED METHODS FOR IDENTIFICATION OF FUNCTIONAL CELL STATES

(71) Applicants: Bartlomiej P. RAJWA, West Lafayette, IN (US); Allison Wooi IRVINE, Oakland, CA (US); ASEDASCIENCES AG, Schindellegi (CH)

(72) Inventors: Bartlomiej P. RAJWA, West Lafayette, IN (US); Allison Wooi IRVINE, Oakland, CA (US)

(73) Assignee: ASEDASCIENCES AG, Schindellegi (CH)

(21) Appl. No.: 18/292,019

(22) PCT Filed: Jul. 26, 2022
(86) PCT No.: PCT/US2022/038327

§ 371 Date: Jan. 25, 2024

Related U.S. Application Data
(60) Provisional application No. 63/225,713, filed on Jul. 26, 2021.

(51) Int. Cl. G01N 33/50; G16B 5/00

(52) U.S. Cl. CPC G01N 33/5014; G16B 5/00

(57) ABSTRACT

Embodiments herein described provide methods for determining phenotypic parameters of cell populations and expressing them in terms of feature vectors that can be analyzed by machine learning classifiers. Embodiments provide methods for determining phenotypic parameters of cell populations in response to an agent. Embodiments provide methods for analyzing the effects of an agent on phenotypic parameters using models trained on effects of reference standards whose in vivo effects are known. Embodiments provide methods for predicting the effect of an agent by the classification by a toxicity classification model. Embodiments provide methods for classifying agents by their effects on phenotypic parameters. Embodiments provide software and computer systems for calculating multiway tensors, reducing their complexity, and analyzing the reduced complexity vectors.

### RELATED APPLICATIONS AND PATENTS

This applications claims priority of and incorporates by reference in its entirety U.S. Provisional Application No. 63/225,713 by the same inventors filed on Jul. 26, 2021.

### FIELD OF THE INVENTION

Embodiments relate to fields of cell assays, physiology, and drug development. Embodiments additionally relate to cytometry and to semi-automated and automated analysis of multi-parametric data, such as cytometry data.

### GOVERNMENT FUNDING

No government funds were used in making the invention herein disclosed and claimed.

### I

Phenotypic compound screening is an important technology for rapid assessment of pharmaceutical compounds. In recent years, a number of techniques have been developed to characterize phenotypic responses of cells to perturbants such as small molecules and biologics. The vast majority of reported work has used traditional bulk biochemical assays, or single-cell techniques based on high-content screening (automated microscopy), as reviewed by, for example, Abraham et al. (“High content screening applied to large-scale cell biology.” Trends Biotechnol. 22, 15-22, 2004) and Giuliano et al. (“Advances in High Content Screening for Drug Discovery.” ASSAY Drug Dev. Technol. 1, 565-577, 2003). These methods often involve large and complex datasets that are difficult to analyze in ways that make the most of the information they provide and, in particular, allow ready comparison of datasets from different screenings. This is especially the case for ultra-high throughput methods for phenotypic compound screening, such as flow cytometry.

The statistical methods that have been implemented for the analysis of complex screening datasets, which can provide means to determine correlations between datasets, all have disadvantages. A technique of this type is provided by Hytopoulos et al. (“Methods for analysis of biological dataset profiles.” US patent app. pub. No. 2007-0135997). Hytopoulos discloses methods for evaluating biological dataset profiles. Datasets comprising information for multiple cellular parameters are compared and identified. A typical dataset comprises readouts from multiple cellular parameters resulting from exposure of cells to biological factors in the absence or presence of a candidate agent. For analysis of multiple context-defined systems, the output data from multiple systems are concatenated. However, Hytopoulos does not outline precise method steps for creating and forming the response profiles. Additionally, Hytopoulos does not provide any working embodiments for practicing the methodology with a biological specimen.

Berg et al. (“Function homology screening.” U.S. Pat. No. 8,467,970) discloses methods for assessing functional homology between drugs. The methods involve exposing cells to drugs and assessing the effect of altering the cellular environment by monitoring multiple output parameters. Two different environments, such as those with different compounds present in the environment, can be directly compared to determine similarities and differences. Based on these comparisons, the compounds can be characterized at a functional level, allowing identification of the relevant cell signaling pathways and prediction of side effects of the compounds. Berg also discloses a representation of the measured data in the form of a “biomap,” which is a very simplified heatmap showing graphically all the measured cellular parameters. Berg is related to measuring biological signaling pathways, rather than physiological responses to stress.

Friend et al. (“Methods of characterizing drug activities using consensus profiles.” U.S. Pat. No. 6,801,859) disclose a method for measuring biological response patterns, such as gene expression patterns, in response to different drug treatments. The response profiles (curves), which are created by exposing biological systems to varying concentration of drugs, may describe the biological response of cells to a particular group or class of drugs. The response curves are approximated using models. The resultant data vectors forming curves or profiles, or their parametric models, can be compared using various measures of similarity. These comparisons form a distance matrix which can be subsequently used in a hierarchical clustering algorithm to build a tree representing the similarity of the profiles.

Moreover, profiling methods of the aforementioned applications to Berg et al. and Friend et al. publications are limited and, in particular, do not provide for using distributions of responses for developing profiles of unknown candidate drugs.

Relatively little work in this area has been performed using flow cytometry, which allows for single-cell analysis of cell states on large populations of cells. See, for instance, Edwards et al. (“Flow cytometry for high-throughput, high-content screening.” Curr. Opin. Chem. Biol. 8, 392-398, 2004, 2004); Oprea et al. (“Associating Drugs, Targets and Clinical Outcomes into an Integrated Network Affords a New Platform for Computer-Aided Drug Repurposing.” Mol. Inform. 30, 100-111, 2011); Robinson et al. (“High-throughput secondary screening at the single-cell level.” J. Lab. Autom. 18, 85-98, 2013) and Sklar et al. (“Flow cytometry for drug discovery, receptor pharmacology and high throughput screening.” Curr. Opin. Pharmacol. 7, 527-534, 2007).

However, the availability of high-throughput fluidic handling systems for cytometry has made it feasible to process an entire 96- or 384-well plate within a few minutes, sampling several thousand cells per well, making cytometry increasingly attractive for high-throughput cell assays. The reports describing the use of high-throughput flow cytometry typically focus on relatively simple assays acquiring from 1 to 5 different variables describing cellular physiology for the analyzed cells. From a mathematical perspective, the data collected in these assays can be described as an array in which the rows store information about individual cells, and the columns describe the measured quantity (e.g., light-scatter characteristics, fluorescence intensity signals, etc.). The measured features can be summarized by a variety of statistics. Most commonly, mean or median fluorescence intensity in a subset of cells of interest is used. After data reduction, the results of an experiment are represented by a vector with elements being the values of the chosen summary statistics. If an experiment involves testing a number of different concentrations of a drug, the final outcome is a 2-D array, with individual columns describing the response curves, for instance by a summary statistic of EC₅₀ value, and the rows encode different drugs. Additional information (e.g., different times of drug incubation) may be represented as added dimensions in the array.

Traditionally, drug response curves are approximated by an a priori mathematical model (such as a sigmoidal log-normal curve, log-logistic curve, Gompertz curve, Weibull, etc.) and the measured drug response information is reduced to a few parameters (or even a single parameter) that describe the curves. The entire process produces a heavily abbreviated compound response summary: typically, a “signature” comprising several EC₅₀ values, that is, values representing a concentration of a compound which induces a response halfway between the baseline and maximum after a specified exposure time.

Such approaches have significant inherent limitations that cannot be easily addressed, if at all. First, they presume the presence of a known mathematical model with appropriate parameterization that describes the behavior of all the tested substances. Second, they presume that a single parameter (EC₅₀) derived from a sigmoidal curve carries all the necessary information about the compound response pattern. And third, they analyze the responses manifested by the measured parameters separately, i.e., in a one-dimensional manner. The data analysis and feature extraction leading to the formation of the response curves is also problematic.

Furthermore, traditional and well-established cytometric data processing relies on a so-called gating process, which involves manual separation of the populations of interest in order to compute simple statistical features of these populations (mean, median, coefficient of variance, etc.). This gating can be highly subjective, and it is difficult to reproduce in an automated setting. Additionally, the computed features are not scaled or standardized to reflect the range of possible biological responses or the precision of the cytometry measurements.

The only exception to this is the tensor analytical approach described by Rajwa et al. in US patent application publication numbers 20160370350 and 20150198584 on Identification of Functional Cell States. These methods produce multiparametric tensor fingerprints that can be compared to one another across different datasets, and accurately characterize flow cytometric data without the need for manual gating. These methods are a substantial advance over the previous methods. They are, however, computationally intensive and can be time consuming.

Embodiments herein described provide further methods for overcoming the significant shortcomings of conventional phenotypic screening methods, in some embodiments, by employing a new methodology for quantifying compound responses. Embodiments described herein provide a number of innovative data acquisition and data processing techniques, which allow meaningful comparisons of multidimensional compound fingerprints without compromising information quality, without a priori assumptions about responses, without the need for manual gating, and with improved speed and reduced requirements for computational resources.

### II

### BRIEF SUMMARY OF SOME ILLUSTRATIVE EMBODIMENTS

A few of the many embodiments encompassed by the present description are summarized in the following numbered paragraphs. These numbered paragraphs are self-referential. In particular, the phrase “in accordance with any of the foregoing or the following” used in these paragraphs refers to the other paragraphs. The phrase means, in the following paragraphs, embodiments herein disclosed include both the subject matter described in the individual paragraphs taken alone and the subject matter described by the paragraphs taken in combination. In this regard, it is explicitly the applicant's purpose in setting forth the following paragraphs to describe various aspects and embodiments, particularly by the paragraphs taken alone and in any and all combinations. That is, the paragraphs are a compact way of setting out and providing explicit written descriptions of all the embodiments encompassed by them individually and in any combination with one another. Applicant specifically reserves the right at any time to claim any subject matter set out in any of the following paragraphs, alone or together with any other subject matter of any one or more of the other paragraphs, including any combination of any values therein set forth, taken alone or in any combination with any other value or values therein set forth. Should it be required, the applicant specifically reserves the right to set forth any or all of the combinations herein set forth in full in this application or in any successor applications having benefit of this application.

### Methods and Analysis

A1. A cell cytometry method for characterizing the effect of an agent on cells comprising:
contacting aliquots of a population of cells with K different control conditions κ, where κ is at least 1, and with I different concentrations i of an agent, where/is at least 1;
measuring P different phenotypic parameters, ψ, in individual cells of each aliquot, where P is at least 2 and, where ψₚ denotes a particular phenotypic parameter, thereby obtaining distributions C_{κ} of the measured values for each control condition κ for each phenotypic parameter ψ and distributions Sᵢ of the measured values for each concentration condition i for each phenotypic parameter ψ, wherein the phenotypic parameters are measured in the individual cells by cell cytometry using a cell cytometer,
generating, for each concentration i of the agent, a response curve feature vector based on the measurements and indicative of the response of the cells to the agent by:
calculating pairwise distances d between the distributions of each control condition C_{κ} and each concentration condition Sᵢ separately for each phenotypic parameter ψ, where

$${d}_{κ , i}^{( ψ )} = D ( {C}_{κ}^{( ψ )} , {S}_{i}^{( ψ )} ) , i = 1 … I , κ = 1 … K$$

and D is a distance function;
calculating for each phenotypic parameter ψ, each concentration i, and each condition κ, a tensor A (a three-dimensional array) comprising all the pairwise distances d_{κ,i}^{(ψ)}

$$A = [ \begin{matrix}{d}_{1 , 1}^{( {ψ}_{1} )} & {d}_{1 , 1}^{( {ψ}_{2} )} & … & {d}_{1 , 1}^{( {ψ}_{p} )} \\ {d}_{1 , 2}^{( {ψ}_{1} )} & {d}_{1 , 2}^{( {ψ}_{2} )} & … & {d}_{1 , 2}^{( {ψ}_{p} )} \\ ⋮ & ⋮ & ⋱ & ⋮ \\ {d}_{1 , I}^{( {ψ}_{1} )} & {d}_{1 , I}^{( {ψ}_{2} )} & … & {d}_{1 , I}^{( {ψ}_{p} )}\end{matrix} , \begin{matrix}{d}_{2 , 1}^{( {ψ}_{1} )} & {d}_{2 , 1}^{( {ψ}_{2} )} & ⋯ & {d}_{2 , 1}^{( {ψ}_{p} )} \\ {d}_{2 , 2}^{( {ψ}_{1} )} & {d}_{2 , 2}^{( {ψ}_{2} )} & … & {d}_{2 , 2}^{( {ψ}_{p} )} \\ ⋮ & ⋮ & ⋱ & ⋮ \\ {d}_{2 , I}^{( {ψ}_{1} )} & {d}_{2 , I}^{( {ψ}_{2} )} & … & {d}_{2 , I}^{( {ψ}_{p} )}\end{matrix} , … , \begin{matrix}{d}_{K , 1}^{( {ψ}_{1} )} & {d}_{K , 1}^{( {ψ}_{2} )} & … & {d}_{K , 1}^{( {ψ}_{P} )} \\ {d}_{K , 2}^{( {ψ}_{1} )} & {d}_{K , 2}^{( {ψ}_{2} )} & … & {d}_{K , 2}^{( {ψ}_{P} )} \\ ⋮ & ⋮ & ⋱ & ⋮ \\ {d}_{K , I}^{( {ψ}_{1} )} & {d}_{K , I}^{( {ψ}_{2} )} & ⋯ & {d}_{K , I}^{( {ψ}_{P} )}\end{matrix} ]$$

calculating for each fiber a_{[κ,ψ]} of the tensor A, a range α between values of distances computed for i=1 and i=I and a maximum rate of change β between values of distances computed for i and i+1, where i takes values from 1 to I-1:

$${α}_{κ}^{( ψ )} = range ( {d}_{κ , 1}^{( ψ )} , {d}_{κ , 2}^{( ψ )} , … , {d}_{κ , I}^{( ψ )} ) , κ = 1 … K \\ {β}_{κ}^{( ψ )} = {max arg}_{i} ( \frac{{d}_{κ , i}^{( ψ )} - {d}_{κ , i + 1}^{( ψ )}}{g ( {S}_{i + 1} ) - g ( {S}_{i} )} ) , κ = 1 … K , i = 1 … I - 1$$

where optional function g(.) provides a transformation ensuring the linearity of the concentration range,
combining, the calculated range α and maximum rate of change β to produce a response curve feature tensor R

$$R = [ \begin{matrix}{α}_{1}^{( {ψ}_{1} )} & {α}_{1}^{( {ψ}_{2} )} & … & {α}_{1}^{( {ψ}_{P} )} \\ {β}_{1}^{( {ψ}_{1} )} & {β}_{1}^{( {ψ}_{2} )} & … & {β}_{1}^{( {ψ}_{P} )}\end{matrix} , \begin{matrix}{β}_{2}^{( {ψ}_{1} )} & {β}_{2}^{( {ψ}_{2} )} & … & {β}_{2}^{( {ψ}_{P} )} \\ {β}_{2}^{( {ψ}_{1} )} & {β}_{2}^{( {ψ}_{2} )} & … & {β}_{2}^{( {ψ}_{P} )}\end{matrix} , … , \begin{matrix}{α}_{K}^{( {ψ}_{1} )} & {α}_{K}^{( {ψ}_{2} )} & … & {α}_{K}^{( {ψ}_{P} )} \\ {β}_{K}^{( {ψ}_{1} )} & {β}_{K}^{( {ψ}_{2} )} & … & {β}_{K}^{( {ψ}_{P} )}\end{matrix} ]$$

vectorizing the tensor R to produce a response curve feature vector r:

$$r = {[ \begin{matrix}{α}_{1}^{( {ψ}_{1} )} , & {α}_{1}^{( {ψ}_{2} )} , & … & {α}_{1}^{( {ψ}_{P} )} , & {β}_{1}^{( {ψ}_{1} )} , & {β}_{1}^{( {ψ}_{2} )} , & … & {β}_{1}^{( {ψ}_{P} )} , \\ {α}_{2}^{( {ψ}_{1} )} , & {α}_{2}^{( {ψ}_{2} )} , & … & {α}_{2}^{( {ψ}_{P} )} , & {β}_{2}^{( {ψ}_{1} )} , & {β}_{1}^{( {ψ}_{2} )} , & … & {β}_{2}^{( {ψ}_{P} )} , \\ {α}_{κ}^{( {ψ}_{1} )} , & {α}_{κ}^{( {ψ}_{2} )} , & … & {α}_{κ}^{( {ψ}_{p} )} , & {β}_{κ}^{( {ψ}_{1} )} , & {β}_{κ}^{( {ψ}_{2} )} , & … & {β}_{κ}^{( {ψ}_{p} )}\end{matrix} ]}^{T}$$

executing a classification model on the generated response curve feature vector to obtain a likelihood that the agent presents a characteristic associated with property of interest.
A2. A method according to any of the foregoing or the following, wherein the phenotypic parameters include any one or more of NFκB, caspase, ERK, SAPK, P13K, AKT, a Bcl-1 family protein, p38, ATM GSk3B and ribosomal S6 kinase.
A3. A method according to any of the foregoing or following, wherein the classification model is a multidimensional regression machine learning model.
A4. A method according to any of the foregoing or the following, wherein the classification model is regularized by an elastic net.
A5. A method according to any of the foregoing or the following, wherein the classification model is trained on response curve feature vectors generated using flow cytometry measurements of cells dosed with known compounds.
A6. A method according to any of the foregoing or the following, wherein the classification model is trained on response curve feature vectors generated using flow cytometry measurements of cells dosed with known compounds having known classification characteristics.
A7. A method according to any of the foregoing or the following, wherein the classification model is a toxicity model trained on response curve feature vectors generated using flow cytometry measurements of cells dosed with compounds of known toxicity characteristics.
A8. A method according to any of the foregoing or the following, wherein the classification model is an inflammation model trained on response curve feature vectors generated using flow cytometry measurements of cells dosed with compounds of known inflammatory or anti-inflammatory characteristics.
A9. A method according to any of the foregoing or the following, wherein the classification model is an inflammation model trained on response curve feature vectors generated using flow cytometry measurements of cells dosed with compounds of known inflammatory or anti-inflammatory characteristics and a counter-screen inflammatory or anti-inflammatory compound is employed in the background cellular environment as an additional control.
A10. A method according to any of the foregoing or the following, wherein the classification model is a DNA damage model trained on response curve feature vectors generated using flow cytometry measurements of cells dosed with compounds of known DNA damage characteristics.
A11. A method according to any of the foregoing or the following, wherein the classification model is a DNA damage model trained on response curve feature vectors generated using flow cytometry measurements of cells dosed with compounds of known DNA damage characteristics and a counter-screen DNA-damaging or DNA-protectant compound is employed in the background cellular environment as an additional control.
A12. A method according to any of the foregoing or the following, wherein the classification model is an antioxidant model trained on response curve feature vectors generated using flow cytometry measurements of cells dosed with compounds of known antioxidant characteristics.
A13. A method according to any of the foregoing or the following, wherein the classification model is an antioxidant model trained on response curve feature vectors generated using flow cytometry measurements of cells dosed with compounds of known antioxidant characteristics and a counter-screen antioxidant or reactive oxygen species-producing compound is employed in the background cellular environment as an additional control.
A14. A method according to any of the foregoing or the following, wherein the classification model is used to classify compounds that are members of a structure activity relationship (SAR) series.

### Controls

Ctr1. A method according to any of the foregoing or the following, where positive control cells are treated with one or more known compounds that trigger a maximal measurable effect on one or more of the measured cell physiology responses.
Ctr2. A method according to any of the foregoing or the following, wherein the negative controls are untreated cells, cells treated with buffer, cells treated with media, or cells treated with a sham compound.

### Cell Cycle

Ccy1. A method in accordance with any of the foregoing or the following, wherein the cell state is a measurement of growth phase of the cells, preferably, a measurement of cell division.
Ccy2. A method in accordance with any of the foregoing or the following, wherein the cell state or cell cycle stage is detected via flow cytometry at single-cell level.
Ccy3. A method according to any of the foregoing or the following, where one of the physiological parameters is the cell cycle.
Ccy4. A method according to any of the foregoing or the following, wherein one of the physiological parameters is cell cycle compartment G1, S, and/or G2/M.
Ccy5. A method according to any of the foregoing or the following, wherein one of the cell cycle compartments is G1, S, and/or G2/M.
Ccy6. A method according to any of the foregoing or the following, wherein all of the physiological responses are measured as a function of cell cycle compartment.
Ccy7. A method in accordance with any of the foregoing or the following, wherein cell cycle phases are measured using fluorescence labels.
Ccy8. A method in accordance with any of the foregoing or the following, wherein cell cycle phases are measured using one or more fluorescent DNA intercalating dyes.
Ccy9. A method in accordance with any of the foregoing or the following, wherein cell cycle phases are measured using one or more of the fluorescent intercalating dyes HOECHST 33342(2′-(4-Ethoxyphenyl)-6-(4-methyl-1-piperazinyl)-1H,3′H-2,5′-bibenzimidazole), DRAQ5™ (1,5-bis{[2-(di-methylamino) ethyl]amino}-4,8-dihydroxyanthracene-9,10-dione), YO-PRO-1 IODIDE (Quinolinium, 4-((3-methyl-2(3H)-benzoxazolylidene)methyl)-1-(3-(trimethylammonio)propyl)-, diIODIDE), DAPI (4′,6-diamidino-2-phenylindole) and CYTRAK ORANGE (derivative of 1,5-bis{[2-(di-methylamino) ethyl]amino}-4,8-dihydroxyanthracene-9,10-dione).
Ccy10. A method in accordance with any of the foregoing or the following, wherein cell cycle phases are measured by immunolabelling of cell cycle-dependent proteins.
Ccy11. A method in accordance with any of the foregoing or the following, wherein cell cycle phases are measured by immunolabelling one or more of cyclins A, cyclin B and cyclin E.
Ccy12. A method in accordance with any of the foregoing or the following, wherein cell cycle phases are measured by immunolabelling one or more phosphorylated histone proteins.
Ccy13. A method in accordance with any of the foregoing or the following, wherein cell cycle phases are determined using genetically encoded cell-cycle dependent fluorochromes such that cell cycle can be monitored using flow cytometry, such as hyper-phosphorylated Rb protein and cycline protein or their phosphorylation states, as described, for instance, in Juan et al. “Phosphorylation of retinoblastoma susceptibility gene protein assayed in individual lymphocytes during their mitogenic stimulation,” Experimental Cell Res 239:104-110, 1998 and in Darzynkiewicz et al. “Cytometry of cell cycle regulatory proteins.” Chapter in: Progress in Cell Cycle Research 5;533-542, 2003.
Ccy14. A method in accordance with any of the foregoing or the following, wherein cell cycle phases are measured by expression of a genetically encoded fusion protein comprising a naturally expressed oscillating protein linked to a fluorescent protein moiety, e.g., cell cycle arrest at G2/M (Cheng et al., “Cell-cycle arrest at G2/M and proliferation inhibition by adenovirus-expressed mitofusin-2 gene in human colorectal cancer cell lines,” Neoplasma 60; 620-626, 2013); regulation of S-phase entry (McGowan et al., “Platelet-derived growth factor-A regulates lung fibroblast S-phase entry through p27kip1 and FoxO3a.” Respiratory Research, 14;68-81, 2013); or identification of live proliferating cells using a cyclinB1-GFP fusion reporter (see Klochendler et al., “A transgenic mouse marking live replicating cells reveals in vivo transcriptional program of proliferation,” Developmental Cell, 16;681-690, 2012).
Ccy15. A method in accordance with any of the foregoing or the following, wherein the cell cycle is altered by an agent.
Ccy16. A method in accordance with any of the foregoing or the following, wherein the cell cycle is altered by a variation in cell culturing method.
Ccy17. A method in accordance with any of the foregoing or the following, wherein the cell cycle is altered by changes in the levels of one or more of the following in the culture medium: glucose, essential and non-essential amino acids, O₂ concentration, pH, galactose and/or glutamine/glutamate.
Ccy18. A method in accordance with any of the foregoing or the following, further comprising detecting the cell state or cell cycle stage in a control population of cells exposed to a plurality of chemicals or agents which are known to perturb the state of the cell cycle.

### Cells

Cls1. A method in accordance with any of the foregoing or the following, wherein the cells are in vitro cultured cells.

A method in accordance with any of the foregoing or the following, wherein the cells are biopsy cells.
Cls2. A method in accordance with any of the foregoing or the following, wherein the cells are live cells.
Cls3. A method in accordance with any of the foregoing or the following, wherein the cells are fixed cells.
Cls4. A method in accordance with any of the foregoing or the following, wherein the cells are a cell line.
Cls5. A method in accordance with any of the foregoing or the following, wherein the cells are characteristic of a naturally occurring healthy cell type.
Cls6. A method in accordance with any of the foregoing or the following, wherein the cells are characteristic of a disease.
Cls7. A method in accordance with any of the foregoing or the following, wherein the cells are characteristic of an inborn genetic disorder.
Cls8. A method in accordance with any of the foregoing or the following, wherein the cells are characteristic of a cancer.
Cls9. A method in according with any of the foregoing or the following, wherein the cells are characteristic of a metabolic disorder.
Cls10. A method in accordance with any of the foregoing or the following, wherein the cells are animal cells.
Cls11. A method in accordance with any of the foregoing or the following, wherein the cells are mammalian cells.
Cls12. A method in accordance with any of the foregoing or the following, wherein the cells are human cells.
Cls13. A method according to any of the foregoing or the following, wherein the cells are germ cells or stem cells, including, pluripotent stem cells.
Cls14. A method in accordance with any of the foregoing or the following, wherein the cells are somatic cells.
Cls15. A method in accordance with any of the foregoing or the following, wherein the cells are stem cells.
Cls16. A method in accordance with any of the foregoing or the following, wherein the cells are embryonic stem cells.
Cls17. A method in accordance with any of the foregoing or the following, wherein the cells are pluripotent stem cells.
Cls18. A method in accordance with any of the foregoing or the following, wherein the cells are induced pluripotent stem cells.
Cls19. A method in accordance with any of the foregoing or the following, wherein the cells are blast cells.
Cls20. A method in accordance with any of the foregoing or the following, wherein the cells are differentiated cells.
Cls21. A method in accordance with any of the foregoing or the following, wherein the cells are terminally differentiated somatic cells.
Cls22. A method in accordance with any of the foregoing or the following, wherein the cells are cardiomyocytes, hepatocytes, neurons or a combination thereof.
Cls23. A method in accordance with any of the foregoing or the following, wherein the cells are one or more of the following: primary cells, transformed cells, stem cells, insect cells, yeast cells, protozoan cells, and/or algal cells, preferably anchorage independent cells, such as, for example, human hematopoietic cell lines (including, but not limited to, HL60, K562, CCRF-CEM, Jurkat, THP-1, etc.); anchorage independent algal cells, such as, for example, Euglenophyta or Chlorophyta, anchorage independent protozoan cells, such as, for example, Plasmodium spp.; or anchorage-dependent cell lines (including, but not limited to HT-29 (colon), T-24 (bladder), SKBR (breast), PC-3 (prostate), etc.).
Cls24. A method in accordance with any of the foregoing or the following, wherein the cells are any one or more of the following: genetically engineered cells, including, but not limited to, for example, cells modified by traditional mutation techniques, recombinant DNA techniques, including, but not limited to, any and all CRISPR and related techniques, cells modified by standard mutagenic techniques, including, but not limited to radiation exposure, and cells having incorporated therein exogenous genetic elements.
Cls25. A method in accordance with any of the foregoing or the following, wherein the cells are any one or more of the following: any primary cell type genetically engineered and/or edited by homologous or non-homologous methods including, but not limited to, CRISPR, wherein the cells can be compared to the normal non-engineered cell type.
Cls26. A method in accordance with any of the foregoing or the following, wherein the cells are any one or more of the following: primary cells comprising a genetic anomaly representative of a genetic or other abnormality, designed for comparison with the normal primary cell and/or other variants thereof.

### Duration

Dur1. A method in accordance with any of the foregoing or the following, wherein cells are exposed to an agent for a plurality of durations or various times, e.g., measuring time course (kinetics) for activation of signaling pathways in cells (see, e.g., Woost et al., “High-resolution kinetics of cytokine signaling in human CD34/CD117-positive cells in unfractionated bone marrow,” Blood, 117; 131-141, 2011). In some embodiments analysis of kinetics is preferred (see Kornblau et al. “Dynamic single-cell network profiles in acute myelogenous leukemia are associated with patient response to standard induction therapy,” Clin Cancer Res, 16;3721-3733, 2010).
Dur2. A method in accordance with any of the foregoing or the following, wherein the cells are exposed to an agent for 0.1, 0.2, 0.3, 0.4, 0.5, 0.6, 0.7, 0.8, 0.9, 1, 2, 3, 4, 5, 6, 7, 8, 9, 10, 12, 14, 15, 16, 18, 20, 22, 24, 26, 28, 30, 35, 40, 44, 48, 52, 56, 60, 66, 72, 78 or more hours or any combination thereof.

### Concentration

Cnc1. A method in accordance with any of the foregoing or the following, wherein a plurality of any one or more or a combination of 2, 3, 4, 5, 6, 7, 8, 9, 10, 11, 12 or more concentrations of an agent is measured.

### Plurality (Number) of Samples

Plr1. A method in accordance with any of the foregoing or the following, wherein a plurality of samples is measured.
Plr2. A method in accordance with any of the foregoing or the following, wherein a plurality of any one or more of and/or any combination of 2, 5, 10, 15, 20, 25, 50, 75, 100, 125, 150, 200, 250, 500, 750, 1,000, 2,000, 3,000, 5,000, 10,000, 15,000, 20,000, 25,000, 50,000, 100,000 or more samples is measured.
Plr3. A method according to any of the foregoing or the following, comprising measuring a plurality of samples disposed in wells of a multiwell plate.
Plr4. A method according to any of the foregoing or the following, comprising measuring a plurality of samples disposed in wells of 96, 384, or 1536-well plates.

### Basic Instrumentation/Methods

Ins1. A method in accordance with any of the foregoing or the following, wherein the responses are measured by cytometry.
Ins2. A method in accordance with any of the foregoing or the following, wherein the responses are measured by flow cytometry.
Ins3. A method in accordance with any of the foregoing or the following, wherein responses are measured by flow cytometry of live cells.
Ins4. A method in accordance with any of the foregoing or the following, wherein responses are measured by flow cytometry of fixed cells.
Ins5. A method in accordance with any of the foregoing or the following, wherein responses are measured by imaging of immobilized cells.
Ins6. A method in accordance with any of the foregoing or the following, wherein responses are measured by fluorimetry.
Ins7. A method in accordance with any of the foregoing or the following, wherein a plurality of two or more response parameters is measured by a multichannel sensor array.

### Signal Processing

Sig1. A method in accordance with any of the foregoing or the following, comprising decorrelating fluorescence signals via linear unmixing of the acquired signals by multiplying the vector of measured values by an inverse of the matrix containing in its columns the spectra of the employed fluorescent species; the said matrix being normalized per column to 1.
Sig2. A method in accordance with any of the foregoing or the following, comprising decorrelating fluorescence signals via linear unmixing of the acquired signals by multiplying the vector of measured values by an inverse of the matrix containing in its columns the spectra of the employed fluorescent species; the said matrix being normalized per diagonal to 1.

### Agents

Agt1. A method in accordance with any of the foregoing or the following, wherein the cells are exposed to a single compound.
Agt2. A method in accordance with any of the foregoing or the following wherein the cells are exposed to two or more compounds.
Agt3. A method in accordance with any of the foregoing or the following wherein one or more of the compounds stimulate a physiological response.
Agt4. A method in accordance with any of the foregoing or the following, wherein the agent may be a genetic agent, e.g. expressed coding sequence; or a chemical agent, e.g. drug candidate.
Agt5. A method in accordance with any of the foregoing or the following, wherein the agent is a drug candidate.
Agt6. A method in accordance with any of the foregoing or the following, wherein the agent is an excipient.
Agt7. A method in accordance with any of the foregoing or the following, wherein the agent is a pharmaceutically active entity.
Agt8. A method in accordance with any of the foregoing or the following, wherein the agent is an industrial or agricultural chemical.

### Physiological Parameters

### MMP

MMP1. A method in accordance with any of the foregoing or the following, wherein mitochondrial toxicity is measured.
MMP2. A method in accordance with any of the foregoing or the following, wherein the loss of mitochondrial membrane potential or integrity is measured.
MMP3. A method in accordance with any of the foregoing or the following, wherein loss of mitochondrial membrane potential or integrity is measured using a fluorescent dye.
MMP4. A method in accordance with any of the foregoing or the following, wherein loss of mitochondrial membrane potential or integrity is measured using one or more of JC-1 (5,5′,6,6′-tetrachloro-1,1′,3,3′-tetraethylbenzimi-dazolylcarbocyanine IODIDE), JC-9 ((3,3′-dimethyl-β-naphthoxazolium IODIDE, MITOPROBE™, Molecular Probes), JC-10 (e.g., derivative of JC-1), DiOC2(3) ((3,3′-diethyloxacarbocyanine IODIDE; MITOPROBE™, Molecular Probes), DiIC1(5) ((1,1′,3,3,3′,3′-hexamethylindodicarbo-cyanine IODIDE; MITOPROBE™, Molecular Probes), MITOTRACKER™ (Molecular Probes), ORANGE CMTMROS (chloromethyl-dichlorodihydrofluorescein diacetate, MITOTRACKER™ ORANGE, Molecular Probes) and CMXROS (1H,5H,11H,15H-Xantheno[2,3,4-ij:5,6,7-i′j′]diquinolizin-18-ium, 9-[4-(chloromethyl)phenyl]-2,3,6,7,12,13,16,17-octahydro-, chloride, MITOTRACKER™ RED, Molecular Probes).

### Cell Viability

Via1. A method in accordance with any of the foregoing or the following, wherein cell viability is measured.
Via2. A method in accordance with any of the foregoing or the following, wherein cell membrane integrity is measured.
Via3. A method in accordance with any of the foregoing or the following, wherein cell viability is determined my measuring membrane integrity.
Via4. A method in accordance with any of the foregoing or the following, wherein loss of membrane integrity is detected using a dye.
Via5. A method in accordance with any of the foregoing or the following, wherein loss of membrane integrity is detected using a dye that enters cells with damaged membranes characteristic of dying or dead cells but does not enter cells with intact membranes characteristic of live cells.
Via6. A method in accordance with any of the foregoing or the following, wherein loss of membrane integrity is detected using a dye that enters cells with damaged membranes characteristic of dying or dead cells but does not enter cells with intact membranes characteristic of live cells, wherein the dye fluoresces on binding to DNA.
Via7. A method in accordance with any of the foregoing or the following, wherein loss of membrane integrity is detected using one or more of the following dyes: PROPIDIUM IODIDE, DAPI and 7-aminoactinomycin D.
Via8. A method in accordance with any of the foregoing or the following, wherein membrane integrity is measured using one or more dyes that cross intact cell membranes and fluoresce upon interacting with intracellular enzymes and remain in the cytoplasm of live cells but diffuse out of cells lacking intact cytoplasmic membranes.
Via9. A method in accordance with any of the foregoing or the following, wherein membrane integrity is measured using one or more dyes that cross intact cell membranes and fluoresce upon interacting with intracellular enzymes and remain in the cytoplasm of live cells but diffuse out of cells lacking an intact cytoplasmic membrane, wherein the dyes are one or more of fluorescein diacetate, CALCEIN AM, BCECF AM, carboxyeosin diacetate, CELLTRACKER™ GREEN CMFDA, Chloromethyl SNARF-1 acetate and OREGON GREEEN 488 carboxylic acid diacetate.
Via10. A method in accordance with any of the foregoing or the following, wherein viability is measured by any one or more of Annexin V, cleaved caspases, and/or caspase activation, including phosphorylation and/or nuclear lamin degradation.

### GLU, ROS, MMP, CMP and Viability

GRC1. A method in accordance with any of the foregoing or the following, wherein one or more of the following physiological parameters is measured: glutathione concentration (“GLU”, “GSH”, or “GTH”), free radicals and/or reactive oxygen species (“ROS”), mitochondrial membrane potential/permeability (“MMP”), cytoplasmic membrane permeability, and cell viability.

### DNA damage, Stress, Inflammation, Metabolism, Apoptosis

DSI1. A method in accordance with any of the foregoing or the following, wherein one or more the following physiological parameters is measured: DNA damage; a stress response signaling pathway constituent; an inflammatory response pathway constituent; a metabolic pathway regulatory constituent or an apoptosis pathway constituent.
DSI2. A method in accordance with any of the foregoing or the following, wherein the stress response signaling pathway constituent SAPK is measured.
DSI3. A method in accordance with any of the foregoing or the following, wherein the inflammatory responses signaling pathway constituent NF-kB is measured.
DSI4. A method in accordance with any of the foregoing or the following, wherein the metabolic pathway regulatory constituent measured is a lipid peroxidase, GSk3B, and/or ribosomal S6 kinase.
DSI5. A method in accordance with any of the foregoing or the following, wherein the apoptotic pathway constituent measured is PI3K, AKT and/or a Bcl-family protein.

### Reference Banks

Rbk1. A method in accordance with any of the foregoing or the following, wherein the known perturbing chemicals or exogenous molecular agents are further sub-grouped based on their known effects.
Rbk2. A method in accordance with any of the foregoing or the following, further comprising creating response tables comprising information about changes in cell viability, mitochondrial toxicity, and at least one additional physiological or phenotypic descriptor at every employed concentration of said compound computed for every stage of cell cycle defined by cell-cycle dependent markers.
Rbk3. A method in accordance with any of the foregoing or the following, wherein feature vectors describing known compounds used to treat a particular disease are grouped into a single defined class or a plurality of defined classes and the compound feature vectors are used as a training set for a supervised machine learning classifier which classifies unknown or not previously characterized compounds into said defined classes.
Rbk4. A method in accordance with any of the foregoing or the following, wherein tensors describing known compounds are grouped into classes on the basis of their off-target responses, such as, side-effects.
Rbk5. The method in accordance with any of the foregoing or the following, wherein feature tensors are used to discover clusters of similar compounds using unsupervised learning.
Rbk6. The method in accordance with any of the foregoing or the following, wherein the feature tensors are vectorized.

### Classification

Cls1. A method for classifying biologically active compounds in accordance with any of the foregoing or the following comprising detecting a plurality of cellular features from a population of cells exposed to said compounds, wherein said features are correlated to morphological properties quantified simultaneously by proportions of light scatter intensity measured at two or more angles.
Cls2. A method in accordance with any of the foregoing or the following, comprising exposing a culture of said population of cells to a plurality of compounds and detecting the physiological response of said population of cells in the presence and absence of said compound.
Cls3 A method in accordance with any of the foregoing or the following, comprising detecting the physiological response of individual cells sampled from said culture.
Cls4. A method in accordance with any of the foregoing or the following, wherein the physiological response is mitochondrial toxicity, which is quantitated in terms of loss of mitochondrial membrane potential or a loss of mitochondrial membrane integrity using one or more fluorescence labels selected from the group consisting of JC-1, JC-9, JC-10, DiOC2 (3), DiIC1 (5), MITO TRACKER® ORANGE CMTMROS, MITO TRACKER® RED CMXROS.
Cls5. A method in accordance with any of the foregoing or the following, wherein the physiological response is overall cell viability, which is quantitated in terms of loss of cellular membrane integrity using one or more fluorescence labels.
Cls6. A method in accordance with any of the foregoing or the following, wherein the fluorescence labels are selected from groups consisting of
dyes which enter the cell interior resulting in a very bright fluorescence (e.g., propidium IODIDE and 7-aminoactinomycin D);
dyes which cross membranes of intact cells and produce fluorescent molecules upon interaction with intracellular enzymes (e.g., fluorescein diacetate, CALCEIN AM, BCECF AM, carboxyeosin diacetate, CELLTRACKER™ GREEN CMFDA, Chloromethyl SNARF-1 acetate, OREGON GREEN 488 carboxylic acid diacetate).
Cls7. A method in accordance with any of the foregoing or the following, further comprising detecting at least one additional physiological or phenotypic descriptor from the group consisting of concentration of glutathione, presence of reactive oxygen species or free radicals.

### Light Scattering

LSg1. A method in accordance with any of the foregoing or the following, wherein a physiological parameter of cell state is measured by light-scattering.
LSg2. A method in accordance with any of the foregoing or the following, wherein a physiological parameter of cell state is measured by laser light-scattering.
LSg3. A method in accordance with any of the foregoing or the following, wherein a physiological parameter of cell state is measured by quantifying the amount of laser light scattered from an individual cell at two or more angles.
LSg4. A method in accordance with any of the foregoing or the following, wherein a physiological parameter of cell state is measured by laser light-scattering, wherein the wavelength of light emitted by the laser is within the range of any one or more of 403-408 nm, 483-493 nm, 525-535 nm, 635-635 nm and 640-650 nm.

### Systems

Sys1. A system for evaluating/comparing biological datasets, comprising a non-transitory computer readable storage medium storing a computer program that, when executed on a computer, causes the computer to perform any of the foregoing or following methods.
Sys2. A system for evaluating/comparing biological datasets, comprising a non-transitory computer readable storage medium storing a computer program that, when executed on a computer, causes the computer to perform any of the foregoing or following methods for characterizing one or more cellular responses to an agent, said method comprising:
measuring by cytometry a plurality of physiological parameters p, of cells in the population which are exposed to a concentration, c, of said agent;
calculating a set of distances between populations and controls for each parameter for the cell population at each concentration; and
compiling a tensor or a set of tensors for each compound (where the tensors contain compound fingerprints); and
compressing the tensors via a feature extraction method to yield an abbreviated compound fingerprint in a form of a vector.
Sys3. A computer system for evaluating/comparing biological datasets, comprising, a non-transitory computer readable storage medium storing a computer program that, when executed on a computer, causes the computer to perform a method for characterizing one or more cellular responses to an agent, said method comprising:
(A) exposing first cell populations to a plurality of concentrations of a first agent, and to a negative control;
measuring by cytometry a plurality of physiological parameters of cells in said populations at each concentration of said first agent and said negative control;
from the measurements compiling one or more tensors indicative of the responses of the cell physiological parameters in said cells of said first populations to said first agent;
compressing said one or more tensors(s) via feature extraction to obtain response curve feature vector(s) (also referred to herein as “response curve vectors”, compound fingerprints”, “fingerprints” and “vector fingerprints”);
(B) exposing second cell populations to a second plurality of concentrations of a second agent, and to a negative control;
measuring by cytometry a plurality of physiological parameters of cells in said second populations at each concentration of said second agent;
from the measurements compiling one or more tensors indicative of the responses of the cell physiological parameters in said cells of said second populations to said second agent;
compressing the tensors(s) via feature extraction to obtain response curve feature vector(s) (also referred to herein as “response curve vectors”, “compound fingerprints”, “fingerprints” and “vector fingerprints”);
(C) calculating a dissimilarity between the first and the second response curve feature vectors to determine one or more differences between the response of the cells to the first and second agents.
Sys4. A computer system for evaluating/comparing biological datasets, comprising, a non-transitory computer readable storage medium storing a computer program that, when executed on a computer, causes the computer to perform a method for characterizing one or more cellular responses to an agent, said method comprising:
measuring two or more cell physiology responses for one or more negative, one or more positive controls and for one or more concentrations of a compound;
calculating a dissimilarity between the distributions of cellular measurements for each positive and negative controls and each of the concentrations in accordance with methods described herein,
thereby to determine the response of the cells to the compound.
Sys5. A computer system for evaluating/comparing biological datasets, comprising, a non-transitory computer readable storage medium storing a computer program that, when executed on a computer, causes the computer to perform a method for characterizing one or more cellular responses to an agent, said method comprising:
measuring two or more cell physiology responses for one or more negative, one or more positive controls and for one or more concentrations of a compound;
selecting subpopulation of cells for the controls and the concentration series by gating the cells in a particular cell cycle compartments and a particular morphological class;
calculating a dissimilarity between the distributions of cellular measurements for each positive and negative controls and each of the concentrations;
thereby to determine the response of the cells to the compound.

### Datasets and Databases

Dbs1. A dataset comprising values for two or more cellular parameters
Dbs2. A dataset comprising measured values for multiple cellular parameters for cells exposed to biological factors in the absence or presence of a candidate agent.
Dbs3. A database comprising compound fingerprint datasets in the form of compound response curve feature vectors.
Dbs4. A database of trusted profiles for the classification of test profiles, where the trusted profiles are compound response curve feature vectors of known and well-characterized compounds.
Dbs5. Datasets may be control datasets, or test datasets, or profile datasets that reflect the parameter changes of known agents. For analysis of multiple context-defined systems, the output data from multiple systems may be concatenated.

### Fingerprints

Fpt1. A drug fingerprint comprising values of multiple cell response parameters.
Fpt2. A drug fingerprint of a genus of compounds, comprising an average of repeated measurements of compound response curve feature vectors.
Fpt3. A drug fingerprint of a genus of compounds, comprising a response curve vector, wherein said vector is derived from the response curve feature vectors of a plurality of compounds.

### III

### BRIEF DESCRIPTION OF THE DRAWINGS

Various features and advantages of the embodiments herein described can be additionally appreciated and better understood in light of the drawings:

FIG. 1 shows an example of cell populations from a series of test wells versus a control well, in a multi-well assay plate for processing by multiparameter flow cytometry. This arrangement illustrates a basic concept underlying the calculation of distance metrics, illustrated graphically in FIG. 2.

FIG. 2 shows representative examples of how distance metric d (QF, Earth Mover's, etc.) is calculated between a control well and each of the test wells, for each flow cytometry parameter p.

FIG. 3A and FIG. 3B are flowcharts showing general process steps for carrying out cell physiology assays.

FIG. 4 is a flowchart showing steps in data analysis using feature classification methods described herein.

FIG. 5 shows a plot of the distance values, between a control and each test concentration of an agent, for a phenotypic parameter, versus the concentration of the agent. For each flow cytometry parameter p, the distance values d are fitted to a model from which two features are extracted: the range f₁ and the point of maximum rate of change f₂.

FIG. 6A-6D show tables of Cell Health Screen risk scores for 40 excipients according to various examples. Column heading key: CM=cell morphology, CMI=cell membrane integrity, ROS=reactive oxygen species, GTH=glutathione, NMI1=nuclear membrane integrity 1, CC=cell cycle, NMI2=nuclear membrane integrity 2, MMP=mitochondrial membrane potential, CHI=Cell Health Index, THR=Target Hit Rate for in vitro assays. THR (i.e., pharmacological promiscuity) is the percentage of targets hit by the compound among all targets tested in the two panels of secondary pharmacology assays.

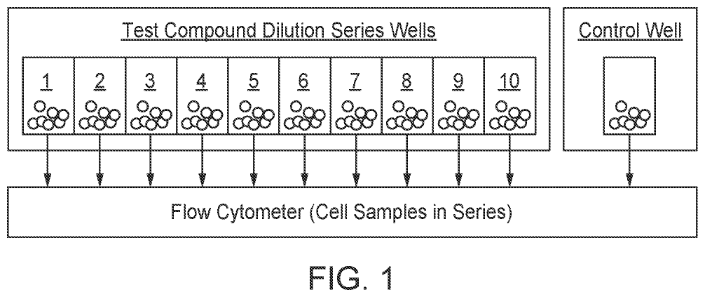

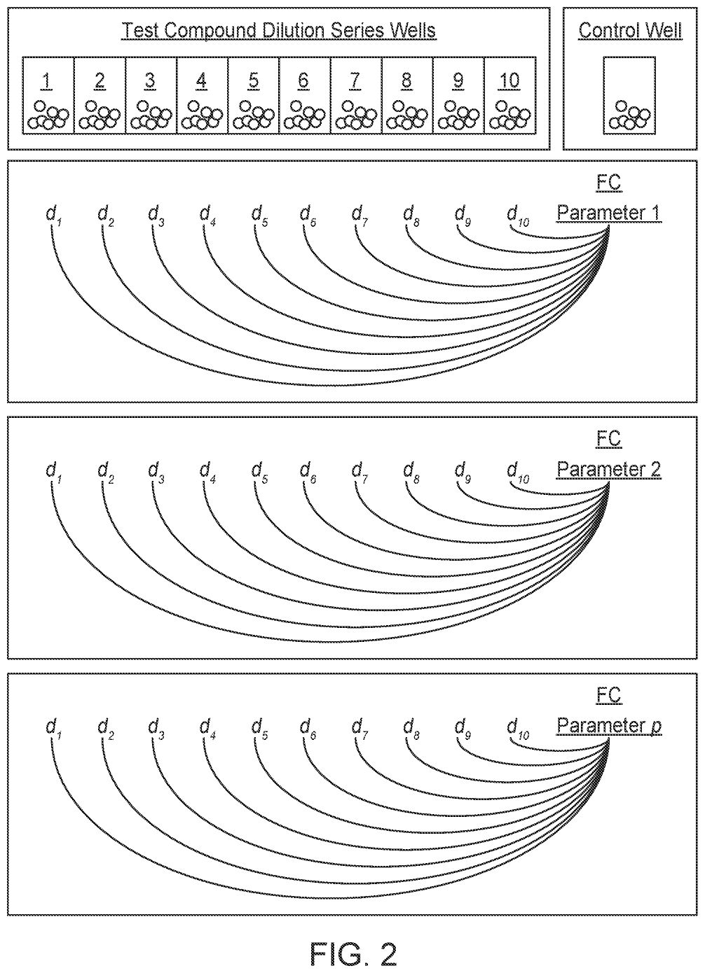

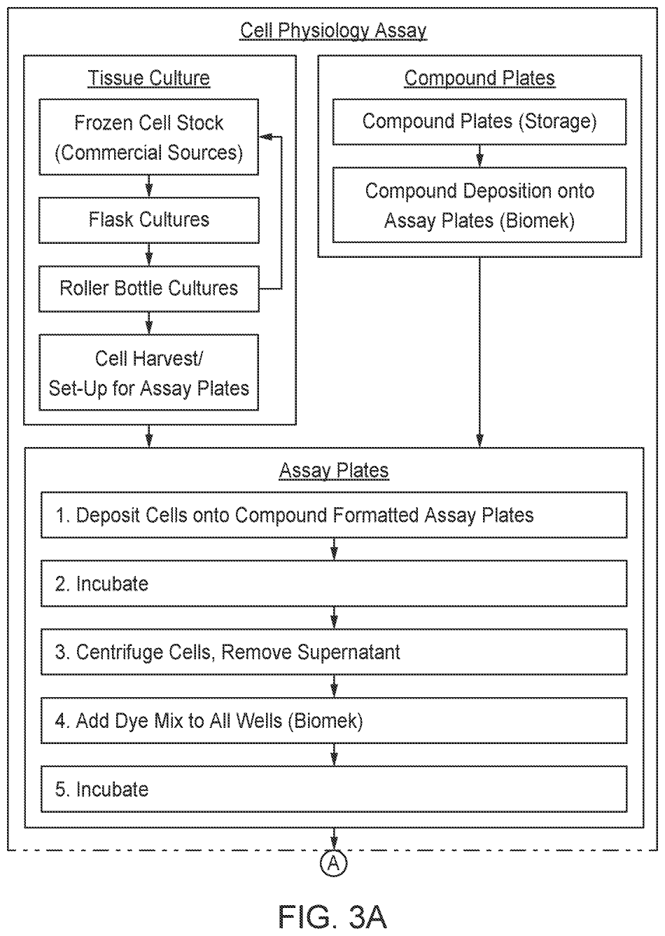

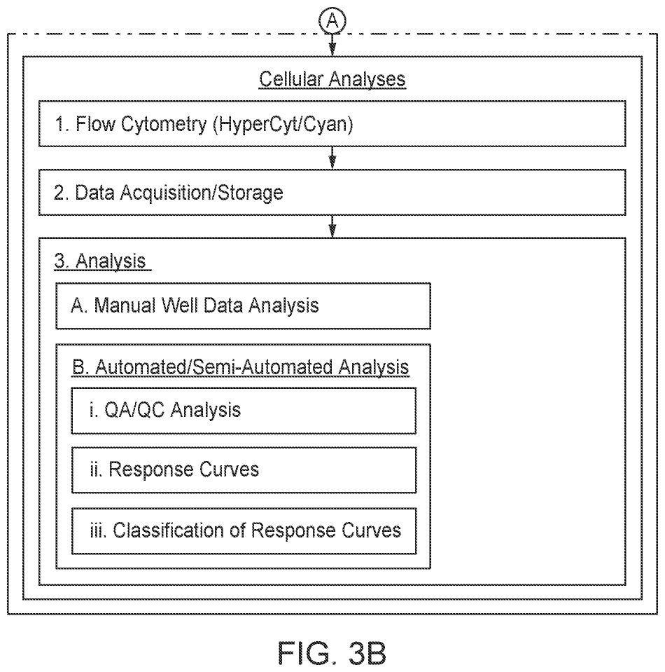

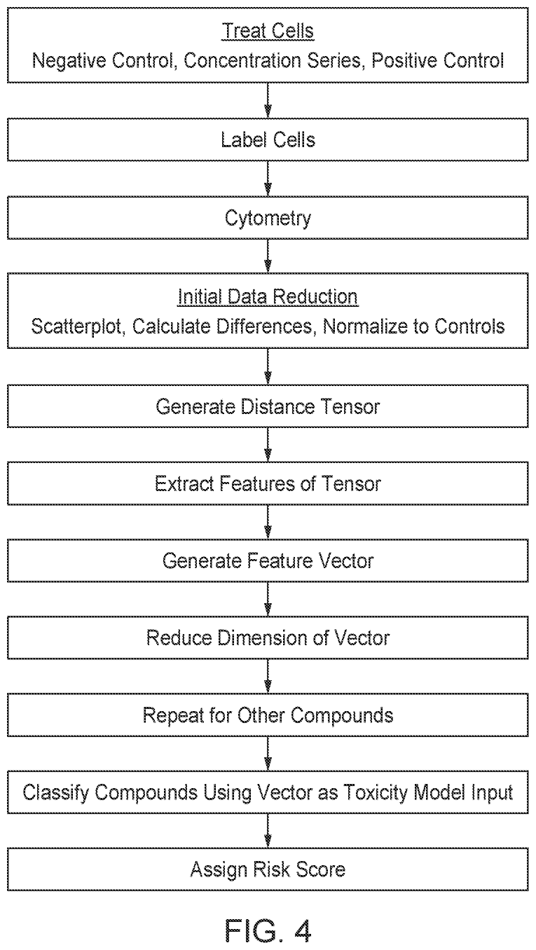

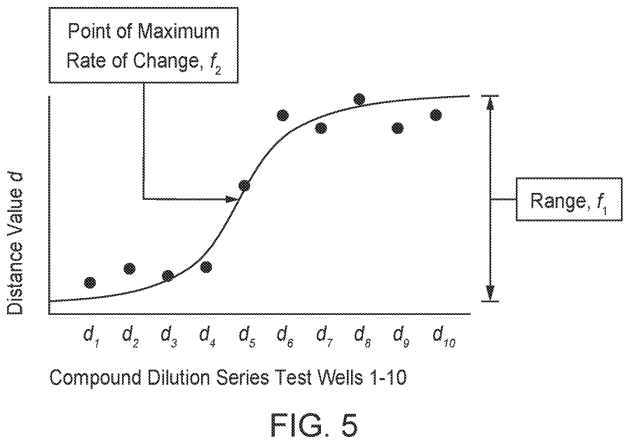

### IV

### GENERAL DESCRIPTION OF A FEW ILLUSTRATIVE ASPECTS AND EMBODIMENTS

Illustrative embodiments of the present invention provide automated, observer-independent, robust, reproducible, and generic methods to collect, compile, represent, and mine complex population-based information, particularly, for instance, cytometry-based information, for example, for quantifying and analyzing physiological responses of cells exposed to chemical compounds, such as pharmaceutical compounds (drugs), toxins, excipients, food ingredients, etc. Various embodiments provide methods for characterizing responses by response curve feature vectors. Illustrative embodiments provide for the use of various statistical measures of distances between distributions in one or more dimensions and measures of dissimilarity between response vectors grouped into response curve feature vectors. In various embodiments, the differences in cellular responses to two (or more) chemical compounds are characterized as the difference between two (or more) response curve feature vectors. Various embodiments provide methods to manipulate, process, store, classify and use the response curve feature vectors.

Aspects and embodiments of the inventions herein disclosed in these respects, and others can be understood from the following description, the Example and Figures, the documents cited herein, and the application disclosure taken as a whole as it would be understood by the person of skill in the arts to which it pertains.

Various aspects and embodiments herein described provide processes for converting raw, multiparametric flow cytometry data into scores. In one illustrative application, the scores represent toxicity risks assigned to small molecule compounds.

Various illustrative aspects and embodiments comprise the following four integrated parts:
(1) Physical screening process using flow cytometry
(2) Feature vector assembly from raw flow cytometry data
(3) Training of a Machine Learning (ML) classifier with training set agents
(4) Application of the ML classifier to classify phenotypes produced by test agents.

Each of these parts is discussed below.

### (1) Physical Screening Process

The physical screening process (data acquisition) involves exposing cells to agents (such as compounds) and measuring various cell phenotypic parameters by flow cytometry or other single cell-based methods. In brief, live cells, such as those of a human leukemia cell line (HL60), are exposed to test compounds in liquid suspension. Many other cell lines can be used. The cells are exposed to each test compound as a dilution series so that dose-dependency patterns of cellular responses (reportable via fluorescent dyes) can be collected by flow cytometry-based detection.

Typically, cells, test compounds, control compounds, and fluorescent reporter dyes are arranged in a multi-well assay plate by using industry-standard automated liquid handling. In the same multi-well plate, certain wells contain cells acting as positive or negative controls. Positive control wells consist of cells exposed to reference compounds known to cause substantial changes in all biological parameters detected by the fluorescent reporting dyes. Negative controls are cell populations that receive no compound treatment, and they are suspended in the same diluent mixture used to create the compound dilution series.

The fluorescent dyes are physiological reporting dyes that produce differential fluorescent signals depending upon cellular biochemical phenomena that occur when living cells experience physiologically stressful conditions. After the compound exposure period, the fluorescent dyes are applied to all wells in the multi-well plate: test compound dilution series wells, positive control wells, and negative control wells.

The fluorescent signals, reflecting cellular biochemical and biophysical phenotypic states, are measured by sending a sample of cells from each plate well through a flow cytometer (approximately 10,000 cells per well). The flow cytometer records values associated with measured fluorescence intensities of each dye simultaneously for each individual cell. Ultimately, the set of cells from each plate well is characterized as a large number of single-cell measurements, called “events” in cytometry vernacular, each event consisting of several values representing each of the fluorescent reporter dyes. Finally, no gating is applied to the flow cytometry data.

The flow-cytometry measurements of cells (events) form several N×P matrices, one matrix per well. In a cell measurement matrix, each of the N rows is associated with a cell, and each of the P columns represents either: a biological parameter (for instance, intensity of a fluorescent dye); a biophysical parameter (such as intensity of laser light scatter registered by a detector and informing cell morphology); or a technical control parameter (such as time of event acquisition). The cell measurement matrices are further processed to provide accessible and actionable data.

### (2) Feature Vector Assembly From Raw Flow Cytometry Data

The creation of simplified feature vectors replaces the tensor decomposition step described in Rajwa et al. in US patent application publication number 20160370350 and 20150198584 on Identification of Functional Cell States.

The cellular stress phenotype caused by a test compound must be represented in a way that includes all the informative parameters (biological and biophysical) across all the concentration steps in the test compound dilution series. One way to achieve this goal is to quantify the difference, for each measured signal, between the distribution of responses formed by a population of cells in a test well and the population of cells in either negative, positive, or both types of control wells.

As mentioned above, the measurements performed in a well can be represented as an N×P matrix. Given access to all the acquired events, for any single measurement parameter pᵢ, i∈(1, . . . P), one can readily estimate an empirical probability mass function Mₚ describing the distribution of all the acquired values placed in column i. Subsequently, one can compare Mₚ, obtained in a particular well and representing a specific type of measurement p, to its counterpart in one (or all) of the control wells.

Let us denote the distribution describing a biological measurement p associated with well w, as M_{w,p}, and the corresponding distribution associated with control well v as M_{v,p}. The value of dissimilarity d (M_{w,p}, M_{v,p}) quantifies and represents the difference between responses observed in an experimental well w and a control well v. Since well w contains a compound of a particular concentration jᵢ, i∈(1, . . . J), it can be said that the dissimilarity d represents the difference between responses observed by examining the control cells and the cells exposed to a compound at this concentration.

The described computation of dissimilarities can be repeated for every compound at every concentration, taking positive and/or negative controls under consideration. At the end of the process, each biological parameter for each compound will be represented by a vector of dissimilarities (d₁, d₂, . . . , d_{J}), where J is the number of tested concentrations in the test compound dilution series. These vectors of dissimilarities are essentially the compound dose-response curves. If two types of control wells are used (“positive” and “negative” controls), with B compounds in J concentrations, it is evident that the process will result in the formation of 2×B×P vectors (curves), each containing J points. In a general case, more than two types of control wells can be employed (for instance, the “positive” control wells may be further divided into wells accounting for different observable biological effects, resulting in a total S number of controls). Therefore, the process of compiling the dissimilarities produces S×B×P vectors of length J.

As described in the original AsedaSciences disclosure, all of these vectors can be arranged into a summary four-way data tensor T, with dimensions S×B×P×J. Alternatively, one can create a series of tensors K, each associated with one of the B compounds. These three-way tensors K have dimension S×P×J:

$$K = [ \text{} \begin{matrix}{d}_{1 , 1 , 1} & {d}_{1 , 1 , 2} & … & {d}_{1 , p , 1} & {d}_{1 , 1 , 2} & {d}_{1 , 1 , 2} & … & {d}_{1 , p , 2} \\ {d}_{2 , 1 , 1} & {d}_{2 , 2 , 2} & … & {d}_{2 , p , 2} & {d}_{2 , 1 , 2} & {d}_{2 , 2 , 2} & … & {d}_{2 , p , 2} \\ ⋮ & ⋮ & ⋱ & ⋮ & ⋮ & ⋮ & ⋱ & ⋮ \\ {d}_{j , 1 , 1} & {d}_{j , 2 , 2} & … & {d}_{j , p , 2} & {d}_{j , 1 , 2} & {d}_{j , 2 , 2} & … & {d}_{j , p , 2}\end{matrix} , \text{} … , \text{} \begin{matrix}{d}_{1 , 1 , s} & {d}_{1 , 1 , s} & … & {d}_{1 , p , s} \\ {d}_{2 , 1 , s} & {d}_{2 , 2 , s} & … & {d}_{2 , p , s} \\ ⋮ & ⋮ & ⋱ & ⋮ \\ {d}_{j , 1 , s} & {d}_{j , 2 , s} & … & {d}_{j , p , s}\end{matrix} ] \text{}$$

According to Rajwa et al in US patent application publication numbers 20160370350 and 20150198584 on Identification of Functional Cell States, the compound tensors K can be further decomposed using various decomposition strategies, such as CP decomposition (see the equation below), Tucker decomposition, CUR-tensor decomposition, and other approaches.

$$K = {\sum }_{r = 1}^{R} {a}_{r} ∘ {b}_{r} ∘ {c}_{r} and$$

$${d}_{jps} = {\sum }_{r = 1}^{R} {a}_{jr} {b}_{pr} {c}_{sr} for$$

$$j = 1 , … , J ; p = 1 , … , P ; s = 1 , … , S$$

The result of the decomposition may be subsequently used in the context of the data analysis pipeline to assess the tested compounds.

This procedure is computationally demanding and can be slow for large datasets. The present application provide a faster and computationally less demanding method in which each tensor K is not decomposed but instead simplified via tensor feature extraction. This process takes advantage of the fact that each of the vectors (K tensor fibers) is physically associated with changes in cellular responses across the J concentrations of a test compound. Therefore, rather than being disconnected, independent values, the entries in the tensor fibers describing readouts at J concentrations are connected in the sense that they form a dose-response curve. Thus, all of the B tensors K can be simplified by reducing or compressing the information content stored in these response curves.

One of the possible approaches to feature extraction involves characterizing each of the dose-response curves (vectors of dissimilarities stored as fibers of tensor K) by two features only: the range of values, forming feature f₁, and the position of maximum change (i.e., a value of j at which the difference between values measured at jᵢ and jᵢ₊₁ is the highest), forming feature f₂.

Therefore, the modified (abbreviated or simplified) tensor K can be represented as R:

$$R = [ \begin{matrix}{f}_{1 , 1 , 1} & {f}_{1 , 1 , 1} & … & {f}_{1 , p , 1} & {f}_{1 , 1 , 2} & {f}_{1 , 1 , 2} & … & {f}_{1 , p , 2} \\ {f}_{2 , 1 , 1} & {f}_{2 , 2 , 2} & … & {f}_{2 , p , 1} & {f}_{2 , 1 , 2} & {f}_{2 , 2 , 2} & … & {f}_{2 , p , 2}\end{matrix} , … , \begin{matrix}{f}_{1 , 1 , s} & {f}_{1 , 1 , s} & … & {f}_{1 , p , s} \\ {f}_{2 , 1 , s} & {f}_{2 , 1 , s} & … & {f}_{2 , p , s}\end{matrix} ]$$

$$where {f}_{1 , p , s} = range ( {d}_{1 , p , s} , {d}_{2 , p , s} , … , {d}_{J , p , s} ) ,$$

$${f}_{2 , p , s} = {max arg}_{j} ( \frac{{d}_{i , p , s} - {d}_{i + 1 , p , s}}{g ( {j}_{i + 1} ) - g ( {j}_{i} )} ) , i = 1 , … J - 1$$

The optional function g(.) provides a transformation ensuring the linearity of the concentration range (e.g. g(x)=log₁₀(x)).

Another example of a feature construction strategy is the computation of parameters associated with the parametric sigmoidal representation of these curves. For instance, one can presuppose a 3-parameter log-logistic model for the dose-response curves and extract the values associated with asymptotes and the inflection point of the curve. Whether the approach to feature construction is parametric (presupposes functional representation of the curve) or non-parametric, the essence of the procedure does not change: each curve with length J is reduced to a set of features G.

After applying these feature extraction (length reduction) approaches, the tensor K for each compound is reduced to a smaller tensor R with dimensions S×P×G. Consequently, this saves the space required for storing the information content because of G<J. At this stage, the smaller tensors R can be further decomposed, as described by Rajwa et al., they can be matricized (turned into matrices), or they can be vectorized (turned into vectors), as described herein.

The following example illustrates an implementation of this procedure. The fibers of tensor R associated with parameter p are concatenated to form a vector of length G×S. Therefore, following this matricization procedure, every compound will be represented by a matrix (two-dimensional array) (G×S)×P. At this stage, the columns of this matrix (representing biological/biophysical parameters) can be used in a machine-learning setting. For instance, a classifier employing only one biological parameter p would use the corresponding column from each compound, with length G×S, as inputs (for either training or classification purposes). Further vectorization (concatenation of matrix columns) changes these matrices into single vectors with G×S×P elements for each of the B compounds. These longer vectors can be used by a classifier designed to take advantage of all measured biological/biophysical parameters instead of only a single parameter p used in the above example.

The choice of dissimilarity/distance computation method does not affect the described procedure. In one embodiment of the process, for instance, for each concentration step in a test compound dilution series, quadratic form (QF) distance is used to calculate the distance between the empirical probability mass functions M associated with a flow cytometry detection parameter in both a test well and a control well in the same plate row. All QF distance values for the dilution series form a dose-response distance curve for that flow cytometry parameter. This is repeated for all flow cytometry detection parameters to produce a multiparametric phenotype signature for the test compound. Finally, as described above, in this illustrative example, all the dose-response QF distance curves are further reduced to two values: the point of the maximum rate of change and the range within which change occurs.

If a sigmoid curve is visualized as approximating this observed response, the point of the maximum rate of change would be approximately the curve's inflection point, and the range would be described by the distance between the low and high “plateaus” of the curve. One additional reduction step may be implemented by choosing only a single type of control per parameter, ensuring that the chosen control types maximize the ability to track changes over the range of parameters. This summarized data reduction process is performed for all flow cytometry parameters, producing a feature vector in which only two values represent each parameter.

Besides QF distance, the method can be implemented using other dissimilarity/distance measures such as but not limited to EMD (Earth Movers Distance, also called Wasserstein distance. and its approximation obtained via Sinkhorn distance), Kolmogorov distance, and symmetrized Jeffrey's divergence. As noted above, the choice of dissimilarity/distance function does not affect the feature computation procedure. Some distances may be better suited to a given practical implementation than others, for instance, in terms of computational time, tuning, interpretability, etc.

Substantially identical procedures can be implemented using two-, three-, and higher dimensionality versions of the probability mass function approximation. This may be especially relevant for cases where there is a significant association or dependence between two or more biological or biophysical parameters. In this setting, instead of computing distances/dissimilarities between 1-D representations of M formed by data obtained by each of the biological/biophysical parameters, the practitioner may compute distances between approximations of 2-D (or n-D, in general) M functions formed by several biophysical/biological parameters. Subsequent parts of the procedure would remain identical, although the length of the final feature vectors would be smaller.

Regardless of the distance function choice, or the dimensionality of M, the final feature vectors quantitatively represent the cellular phenotype caused by a test compound.

### (3) Training the ML Classifier

The next step in certain aspects and embodiments of the inventions herein described is to classify the feature vector. In some aspects and embodiments, this can be done using two interconnected tools: (1) a training set, which is a set of known chemical compounds used to provide examples illustrating how the distinct outcome classes (for instance, high versus low toxicity risk) look in the feature space; (2) a supervised ML classifier, which has the ability to assign the new feature vectors into defined classes using estimation of the class boundaries computed from the training set.

Before describing how the ML classifier itself is designed, it is conceptually helpful to understand how a training set is used to train a classifier and why the training set's quality is essential. In the context of a supervised ML classifier, the purpose of a training set is to provide example instances of the known outcome classes among which the classifier is intended to discriminate. Each instance has two characteristics: (1) known outcome class (for our purposes, drugs with known effects, such as safety histories indicating either high or low toxicity risk); (2) descriptive data in the same feature space that the classifier will use to estimate outcome probability, such as, for example, cellular phenotypic data associated with drug exposure. These instances of known outcome class are employed to tune the classifier, enabling it to predict outcome class membership probability from inputs that are based on measured characteristics of a tested instance. If a training set contains a sufficient number of instances associated with historically known outcomes (“ground truth”) and their associated measured features, the properly trained classifier may be able to estimate the outcome for a test instance given access to measured features acquired in an analogous manner. Of course, this approach works if the classes are separable according to the measured features. If the feature distributions overlap too much between classes, classifier separation of classes may not be clear or may not even be possible.

An illustrative example in this regard involves using a cellular stress phenotype indicative of toxicity caused by a chemical compound and detected through flow cytometry as the feature set communicating the measurement input. Based on this input, the ML classifier should predict the likelihood that a compound has high toxicity risk. This “high toxicity risk” can translate to a drug candidate failing because of safety concerns (poor animal trial performance, severe side effects in human clinical trials, withdrawal from the market, etc.) or an industrial/agricultural chemical causing safety problems through human exposure.

In this example, described in greater detail in the Examples below, a training set was assembled from 300 known compounds drawn from on-market pharmaceuticals, withdrawn drugs, research compounds, and a few industrial/agricultural compounds.

All the compounds were assigned to one of two historically known outcome classes: (1) known toxicity and thus high expectation of acute cell stress—the “yes”/“positive” class, and (2) (no known toxicity and thus low expectation of acute cell stress—the “no”/“negative” class. Assignment was based upon manually curated information gathered from the scientific literature, clinical trial results, and known commercial histories.

For many compounds that have known toxic side effects, the scientific research literature directly documents cellular effects, e.g., mitochondrial dysfunction, reactive oxygen species generation, etc. These compounds serve as perfect training instances for one outcome type (high risk) to be predicted. Compounds that have no known toxic side effects are more difficult (but not impossible) to affirmatively document. For examples of this outcome type (low risk), the determination was based on the compound's development history, such as clinical trials, or its commercial history after going on-market, etc. If the scientific literature contained no detectable evidence of cytotoxic mechanisms and the development/commercial history of the compound was otherwise clean with regard to safety, it was assigned to the “no” or low-risk class.

After these “yes/no” outcome assignments, all 300 compounds were physically processed by flow cytometric methods (the Cell Health Screen described in the Examples below), to produce associated feature vectors as described in the “Physical screening process” section above. At this point, every compound in the training set has two data types associated with it: the binary assignment to the historically known outcome (“ground truth”) and the empirical measurement of cellular stress phenotype (feature vector). If one visualizes the feature vectors of the two outcome-based groups of compounds, it is reasonable to expect that each group forms a cloud in the feature space containing the cellular stress measurements. These clouds may overlap; however, provided that the external descriptive information was curated well enough for the “yes/no” outcome assignments and provided that there is a functional relationship between cellular stress and a compound's risk of safety problems (i.e., the two data clouds do not entirely overlap), the training set should be sufficient to provide a template for future prediction by the ML classifier. Given cellular stress measurement from an unknown compound, the trained ML classifier delivers a class assignment and can also estimate the probability with which the new measurement belongs to either of the two classes.

One aspect worth noting before going into the details of how the classification step works is that, at best, training sets can and, in most cases, should be designed to comport with the nature of the screens that will be used and the predictive outcome desired. For instance, outcome assignments in this example were not made on the basis of public safety information without searching the scientific literature for documentation of known cellular toxicity mechanisms. The Cell Health Screen used in this example is designed to predict toxicity risk arising from cellular energy metabolism, ion flux, reactive radical formation, and similar mechanisms that cause acute cellular stress rapidly via physiological phenomena that are detectable with commercially available fluorescent dyes. Other types of chemical safety problems, such as teratogenic effects or hormonal disruption, are not detected in this physical screen design. This design choice was driven by the fact that cellular effects, such as mitochondrial dysfunction and ion imbalances, are known to underlie several more common adverse safety events such as liver damage, cardiac dysfunction, and neuropathies. Teratogenic effects and hormonal disruption are problems that arise more often in the context of pregnancy, child development, or cancer potentiation; as such, these are also important risks to detect, but they need to be addressed by a separate design process. Consequently, this training set was curated so that it would not inadvertently train the classifier with outcome types that cannot be informed by our screen's measurement parameters. Similar considerations apply to the design of other training sets.

### (4) Applying the ML Classifier to Classify Test Compounds

By way of illustration, the classifier discussed herein, implemented for analysis of the cell-based screen data described above and in greater detail in the Examples, uses a logistic regression model regularized by an elastic net. The employed logistic model is multidimensional (i.e., it uses multiple regression) as it must simultaneously utilize information from each of the flow cytometry detection parameters, which are encoded in the phenotypic feature vector for each test compound, as described above. To visualize what is happening, imagine a simple, one-dimensional logistic regression. To train the classifier for one dimension or one detection parameter, the feature values for that detection parameter from all 300 training compounds in this example are applied to one logistic regression. A logistic model is optimized by finding parameters for a curve that most effectively separates the populations of feature values from the “yes” and “no” training classes. For a multidimensional model, this process is performed computationally for all detection parameters simultaneously, resulting in a model that finds the most parsimonious separation of the “yes” and “no” training set compounds along all measurement axes.

Additionally, the model is regularized to minimize the potential detrimental influences of a large number of predictors (measurement features used as input). These possible detrimental effects are: 1) predictive signals may be unevenly distributed among input features so that most predictive power is concentrated in a subset of the features; 2) some of the predictors may be correlated and thus not entirely independent. In elastic net regularization, two types of model penalties are implemented: L₁ (LASSO regression) and L₂ (Ridge regression). These regularizations penalize the size of parameter estimates in order to completely eliminate some of them (LASSO) or shrink them continuously towards zero (Ridge). Specifically, LASSO penalizes the sum of their absolute values (L₁ penalty), and Ridge regression penalizes the sum of squared coefficients (L₂ penalty). The advantage of the elastic net is that it combines L₁ penalty, suitable for a situation in which only a few predictors actually predict the response in a meaningful fashion, and L₂ penalty, which is more appropriate for a case of multiple predictors providing similar predictive value.

Therefore, in a preferred embodiment, the problem is formulated as a binary decision with two class-conditional probabilities:

$$log \frac{Pr ( {y}_{i} = + 11 x )}{Pr ( {y}_{i} = - 11 x )} = X β where ( - 1 , + 1 ) are class labels .$$

The use of elastic net regularization leads to the model:

$$\hat{β} = arg {min}_{β} \{ \frac{1}{2 n} {‖ Y - X β ‖}_{2}^{2} + {λ}_{2} {‖ β ‖}_{2}^{2} + {λ}_{1} {‖ β ‖}_{1} \} .$$

The classifier is trained by a method known as repeated cross-validation and grid search for β and the values controlling the LASSO and Ridge penalties (λ₁ and λ₂). The optimally fit model then becomes the classification tool allowing calculation of the likelihood that a phenotypic feature vector from any compound can be assigned to the “yes” (high cell stress) class. Subsequently, for any test compound, the final risk score, or Cell Health Index (CHI), is the probability with which the test compound's phenotypic feature vector can be assigned to the “yes” class according to the boundary between the classes described by the ML model.

In addition, a series of unidimensional classifiers (simple regressors) are trained and applied to the detection parameters separately, calculating the probability of “yes” class assignment if only data for each flow cytometry parameter were considered in isolation. These single parameter classifications produce an additional “fingerprint” of scores that can be interpreted as indicating the relative ability of each parameter to form a prediction aligned with the final score. This information may indicate the biological relevance of an individual predictor. However, note that the predictivity of the individual parameters cannot be assumed a priori to be equal. Moreover, the elastic net regressor can provide a ranking of features based on their contribution to the trained classifier. This ranking provides information about a predictors' “quality” and relevance in a statistical sense.

Although elastic net regression is the preferable classification approach in the current implementation of the data analysis pipeline, it is not the only classifier capable of delivering the expected results. Other classifiers that may fit in the proposed pipeline include support vector machines (SVM), neural networks (NN), or Bayesian approaches.

It is also important to recognize that the binary problem formulation is not the only framework in which the described process may be executed. As mentioned before, one can design a number of controls reflecting several feasible phenotypes. Each of these phenotypes may be associated with a class, leading to a multiclass classification problem utilizing (K−1)-logits.

$$log \frac{Pr ( {y}_{i} = k \vert x )}{Pr ( {y}_{i} = K \vert x )} = X {β}_{k} for k = 1 , 2 , … , K - 1$$

This setting can be subsequently tackled using multinomial regression with the multiclass elastic net penalty or another multiclass classification method.

### Cytometry

Methods of various embodiments described herein are suitable for analysis of complex multi-parametric data on individual cells in cell populations, as determined by cytometry. Cytometric instruments and techniques, summarized herein (e.g., flow cytometry and imaging cytometry) allow for the simultaneous measurement of multiple intrinsic features (e.g., light scatter, cell volume, etc.) or derived features (e.g., fluorescence, absorption, etc.) of individual cells. Light scatter and fluorescence represent the most commonly utilized measurements for current cytometric applications. Fluorescence measurements can be performed using either “intrinsic” fluorophores naturally present in cells (such as, for example, porphyrins, flavins, lipofuscins, NADPH), fluorophores genetically engineered for specific expression (e.g., GFP, RFP, etc.), or fluorescent reporters which target specific epitopes or structures in or on various cell types (e.g., fluorophore conjugated antibodies, aptamers, phage display, or peptides, or reporters that are converted from non-fluorescent to fluorescent states by specific enzymes in or on cells).

Cytometric techniques useful in embodiments herein described utilize living cells (e.g., using probes which report on aspects of cell physiology, such as, for example, mitochondrial membrane potential, ROS, glutathione content, or a combination thereof). Cytometric techniques useful in some embodiments employ cells that are fixed and permeabilized to allow transport of fluorophores, conjugated reporters, etc., into the cytoplasm and/or the nucleus.

### General Methods for Cellular Assays Using Flow Cytometry

General methods useful for cytometry in accordance with various aspects and embodiments herein described are described below.

### Culture of Anchorage Independent Cells

Cells and methods suitable for activity assays and analysis by flow cytometry that are well known and routinely employed in the art can be employed in carrying out embodiments of inventions described herein.

Cells for assays may be obtained from commercial or other sources. Cells derived from human cancer can be used, such as those from leukemias (e.g., HL60 cells currently used in the cell physiology assay), which grow unattached to the culture vessel. Cells generally can be stored in liquid nitrogen in accordance with standard cell methods. Frozen cells are rapidly thawed in a 37° C. water bath, and cultured in stationary flasks in pre-warmed fresh tissue culture medium in a 37° C. tissue culture incubator. Tissue culture media typically is replaced daily for the first 2-4 days in culture to dilute out the DMSO.

Once growth is established in stationary flasks (cell number and viability is monitored using a Vi-CELL™ cell counter), aliquots of cells can be removed for freezer storage (these early passage cells are only used for backup). In addition, these cells can be used to establish roller bottle cultures needed to have sufficient cell numbers for plate assays. Cells growing in flasks are placed in roller bottles at relatively high cell concentration (˜10⁶ cells per ml in 200 ml fresh tissue culture medium) and cultured in a tissue culture incubator. Initially, roller bottle cultures typically are fed by the addition of a fresh tissue culture medium. Once growth is established, cells are removed as needed to maintain cells at a concentration of 0.5-1.5×10⁶ viable cells/ml. Many cell types adapt to roller bottle cultures slowly and need weeks to successfully adapt to these types of cultures. Successful roller bottle adaptation is evidenced by continuous high viability (˜95%) and consistent growth rates (measured using doubling time). When successfully adapted, stocks of cells are frozen (in 50 ml sterile tubes containing sufficient cells to initiate one new roller bottle culture) in order to maintain cells used for assays at a similar low passage number (details below). Cells maintained in roller bottles are harvested for assay plates, centrifuged, and resuspended in fresh tissue culture media at appropriate cell concentration for the assay to be performed (cell number and viability measured and recorded for each harvest).

As indicated above, roller bottle adapted cells can be frozen for future use, to maintain similar low passage number cells for all plate assays. Roller bottle cell cultures can be maintained for one month before switching to a new lot of low passage frozen cells. During the month of routine use, one tube of frozen cells typically is thawed and re-established to roller bottle culture. Once successfully adapted to roller bottle culture (as above) the newest lot of cells usually is first evaluated for assay performance (see “Cross-Over” studies, below), before this lot of cells is used in plate assays. Establishing frozen cells to roller bottle culture and testing routinely takes 10 to 21 days.

Cells generally are routinely tested at multiple steps in the culture process for mycoplasma contamination. These include initial flask cultures, roller bottle adapted cells, and each tube of frozen cells (tested before each “Cross-Over” study). Mycoplasma testing can be provided by an external. certified testing company, typically using a PCR-based assay.

### Compound Storage and Compound Assay Preparations

Test compounds are generally obtained as 10 mM stocks in DMSO deposited in 96-well plates. Compound plates are stored sealed, protected from light, at either −20° C. or −80° C., depending upon storage period. For compound assays, stock solutions are diluted and deposited into assay plates using a liquid handling system. All dilutions and compound deposition into assay plates are performed the same day as the assay is performed.

Reproducibility of assays should be assessed using test compounds. A set of 16 compounds that have well documented impacts on specific cell physiological measurements have been used to test the reproducibility of cell physiology assays. These compounds are stored, as above, as 10 mM assay solutions in DMSO in 96-well plates. For “Cross-Over” studies, the 16-compound set is used to compare the physiological responses of the newly thawed and roller bottle adapted cells with current lots of production cells.

### Cell Physiology Assays

For cell physiology assays it can be convenient to use 2 sets of 384 well plates to measure the impact of compounds on ten or more cellular response parameters. For both sets of plates, compound dilutions are first deposited into wells, and then 1×10⁵ assay cells are added to each well. Compounds are routinely run with duplicate compound dilution sets on the same plate to measure reproducibility of responses. After thorough mixing, plates are sealed (using an O₂/CO₂ permeant seal) and placed into a 37° C. tissue culture incubator for varying periods of time (typically 4 hrs). Plates are then centrifuged, half the supernatant fluid is removed, and this volume is replaced by the same volume of the appropriate dye mix (for plate A, the dye mix may include Monobromobimane, Calcein AM, MitoSOX™ Red, and SYTOX™ Red; for plate B, the dye mix may include Vybrant™ DyeCycle™ Violet (live cell cycle), JC-9 (mitochondrial membrane potential), and Propidium iodide), followed by mixing. Plates are returned to the tissue culture incubator for 10 (plate A) or 30 (plate B) minutes, followed by a mixing step. Samples are then immediately processed on a flow cytometry system.

The data from positive and negative control wells on each row are used to calculate the responses as described in greater detail herein. The positive control compounds used for plate A and B are different, and they are designed to provide a unique “signature” (“finger print”) in the cell responses measured in plate A or B, using the disclosed embodiments.

### High Throughput Flow Cytometry

In a variety of assays, the flow cytometer is set up using a standard procedure on each day that plates are assayed. Set up includes flow instrument QA/QC using fluorescent beads, which are used to check each detector (PMT) for consistent performance. Each well of a 384 well plate is then sequentially sampled using a 3 or 5 second sip time (plate A versus plate B), followed by a 0.1-second air bubble between samples. The sample stream flows through the flow cytometer in a continuous fashion, sampling a complete plate in 40 to 50 minutes (plates A and B, respectively).

The flow cytometry data files are subsequently processed to identify individual well data, and they are then stored on a server as the list mode data (LMD) for each individual assay well. Separate files, each consisting of a spreadsheet that matches each plate, provide a map of assay well contents so that test compounds and controls can be identified.

### QA/QC Analysis

Both plates (A and B) contain negative controls (untreated samples), and positive controls (samples treated with known compounds chosen to stimulate a positive response, which can be a maximal response). The dissimilarity between positive controls and negative controls does not define in this assay the possible range of responses. However, it defines a unit of response. During the time of sample acquisition for an entire plate, the dissimilarity between positive and negative controls may change owing to deteriorating physiological conditions in the plate (change in temperature, O₂, etc.). This is why a certain minimum level of dissimilarity for every pair of controls is expected. For each positive and negative control within a single row, the disclosed embodiments determine the QF distance between the positive and negative populations for each dye response individually. The disclosed embodiments then plot the change in QF distance from the beginning (row A) to the end of the plate (row P).

### Cytometer Instrumentation

Current flow cytometry instruments are equipped with multiple lasers and multiple separate fluorescence detectors that can simultaneously quantitate many fluorescence signals plus intrinsic optical features originating from individual cells. Thus, cytometric techniques and instruments such as those illustratively described below allow measurement of thousands to millions of cells in a sample. The resultant extremely large data sets present a significant challenge to the presently-employed cytometry data processing and visualization methods. These challenges are handled effectively by methods described herein.

Modern cytometers typically are designed for simultaneously detecting several different signals from a sample. A variety of cytometers are available commercially that can be used in accordance with methods described herein. A typical instrument includes a flow cell, one or more lasers that illuminate the flow cells through a focusing lens, a detector or light passing through the flow cell, a detector for forward scattered light, several dichroic mirror—detector arrangements to measure light of specific wavelengths, typically to detect fluorescence. A wide variety of other instrumentation often is incorporated in commercial instruments.

In typical operations, the laser (or lasers) illuminates the flow cell (here “flow cell” refers to an optical chamber in the sample path) and the cells (or other sample) flowing through it. The volume illuminated by the laser is referred to as the interrogation point. Flow cells are made of glass, quartz and plastic, as well as other material. Although lasers are the most common source of light in cytometers, other light sources can also be used. Almost all cytometers can detect and measure a variety of parameters of forward-scattered and side-scattered light, and several wavelengths of fluorescence emission as well. Detectors in these instruments are quite sensitive and easily quantify light scattering and fluorescence from individual cells within very short periods of time. Signals from the detectors typically are digitized and analyzed by computational methods to determine a wide variety of sample properties. There are many texts available on flow cytometry methods that can be used in accordance with various aspects and embodiments of the inventions herein described. One useful reference in this regard is Practical Flow Cytometry, 4th Edition, Howard M. Shapiro, Wiley, New York (2003) ISBN: 978-0-471-41125-3.

### Spectral Unmixing of Flow Cytometric Signals

Since the signals emitted by the functional fluorescence labels are measured by a series of detectors in a cytometry system (flow- or image-based), the detection systems are prone to spectral cross-talk. As a result, the intensities of individual fluorochromes cannot be measured directly to the exclusion of other fluorochromes. In order to minimize or eliminate noise due to spectral cross-talk, all of the collected signals can be modeled or processed as linear mixtures. The signal mixture for each measured cell is decomposed into approximations of individual signal intensities by finding minimal deviance between the measured results and approximated compositions which are formed by multiplying the estimator of the unmixed signal with the mixing matrix. The mixing matrix (also called “spillover matrix”) describes the n-band approximation of fluorescence spectra of the individual labels (where n is the number of detectors employed in the system). An application of a minimization algorithm allows to find the best estimation of the signal composition. This estimation provides information about the abundances of different labels. In the simplest case, if the measurement error is assumed to be Gaussian, the unmixing process may be performed using ordinary least-squares (OLS) minimization.

### Variance Stabilization

Variance stabilization (VS) is a process designed to simplify exploratory data analysis or to allow use of data-analysis techniques that make assumptions about data homoskedasticity for more complex, often noisy, heteroskedastic data sets (i.e., random variables in the sequence have different finite variance). VS has been routinely widely applied to various biological measurement systems based on fluorescence. It is an important tool for analysis of microarrays.

In the context of flow cytometry and in microarray analysis, log transformation has traditionally been used. However, modern approaches, for example, in the context of microarray analysis are known. For example, see Rocke et al. (Approximate variance-stabilizing transformations for gene-expression microarray data.” Bioinformatics, 19, 966-972, 2003) and Huber et al. (“Variance stabilization applied to microarray data calibration and to the quantification of differential expression.” Bioinformatics, 18, S96-S104, 2002). Huber describes the use of a hyperbolic arcsine function in variance stabilization. In the context of flow cytometric data analysis, Moore et al. (“Automatic clustering of flow cytometry data with density-based merging,” Adv Bioinformatics, 2009) uses logical transformation. Bagwell (“Hyperlog-a flexible log-like transform for negative, zero, and positive valued data.” Cytometry A. 64 (1): 34-42, 2005) describes the use of hyperlog transformation in the analysis of output from flow cytometers.

In an embodiment of the present invention, in contrast, hyperbolic arsine technique (generalized logarithm) with an empirically found parameter is used in variance stabilization.

$$glog ( x ) = asinh ( x ) = log ( x + \sqrt{{x}^{2} + 1} )$$

$$glog ( x , a , b , c ) = asinh ( ax + b ) + c$$

### Comparisons

Certain embodiments described herein provide methods involving a comparing step, wherein the distribution of the unmixed signal intensities is compared to the distribution of the unmixed signals originating from controls or other test data. Depending on the comparison method applied, the distributions may be first normalized by dividing every distribution by its integral.

The comparing step may involve compilation of response curve feature vectors containing information about dissimilarities between cellular populations such as before and after treatment. The dissimilarities are computed as distances between signal distributions of the treated population of cells, untreated populations (“negative” or “no effect” controls), and populations treated with a mixture of perturbants designed to maximize the observable physiological response (“positive” or “maximum effect” controls).

In order to standardize the result and render it unaffected by experimental variability, the measured dissimilarity can be expressed in units equal to mean dissimilarity between positive and negative controls.

Various measures of dissimilarity or distance can be applied, including (but not limited to): Wasserstein metric, quadratic-form distance (QFD), quadratic chi-distance, Kolmogorov metric, (symmetrized) Kullback-Leibler divergence, etc. In the preferred implementation, the methods and algorithms of the instant invention use Wasserstein metric or quadratic chi-distance.

In illustrative methods, the abundance distributions are typically compared in one dimension. However, some labels are encoded by two related signals (for instance, JC-1, the mitochondrial membrane potential label that emits fluorescence in two separate channels). In this case, a 2-D dissimilarity measure between distributions is computed. Finally, it may be preferable to compute 2-D or 3-D dissimilarity measures by utilizing multidimensional distributions based on morphology-related measurements (obtained via light scatter) and an abundance (computed from the fluorescence signal). A variety of distances or dissimilarity measures, assuming that they are easily generalizable to multiple dimensions, may be used. For instance, routine methods based on the Wasserstein metric or the QFD may be used in this context, but not the Kolmogorov metric.

### Analysis

Cytometric multi-parametric data can be expressed as tensors and the comparisons between controls and tested samples can be described by response curve feature vectors. A tensor is a multidimensional array and can be considered as a generalization of a matrix. A first-order (or one-way) tensor is a vector; a second-order (two-way) tensor is a matrix. Tensors of order three (three-way) or higher are called higher-order tensors.

Biological measurements performed in a single-cell system individually for every cell in a population form a distribution. A distance between a distribution of measurements performed on cells exposed to a presence of a compound, and a distribution of measurements performed on cells not exposed to the compound can be expressed by a single number (scalar value). The cells may be exposed to a number of different drug concentrations, and a biological measurement can be performed for each of these exposure levels. Such an experiment produces a series of values that can be expressed as a vector (e.g., a one-way tensor). If multiple biological parameters are measured, the results can be arranged in a two-way tensor (or a matrix), in which every column contains a different measured parameter and every row describes a different concentration of the compound.

This arrangement of data can be expanded further. Attempts to measure the distances between the distributions of measurements obtained from treated cells and a distribution of measurements collected from population of cells exposed to another compound, may group the results into another matrix. For instance, it may be beneficial to measure dissimilarity between cells treated with one compound and another group of cells treated with a different and well characterized compound that creates an easy to observe effect serving as a positive control.

The foregoing analysis can be stated in general terms in the form of the following equation and operations herein referred to as

### General Method and Equations

The cytometry data represent aliquots of a population of cells with K different control conditions κ, where K is at least 1, and with I different concentrations i of an agent, where I is at least 1. The measurement involves obtaining P different phenotypic parameters, ψ, in individual cells of each aliquot, where P is at least 2 and, where ψₚ denotes a particular phenotypic parameter (p=1 . . . P). The measurement allows obtaining distributions C_{κ} of the measured values for each control condition κ for each phenotypic parameter ψ, and distributions Sᵢ of the measured values for each concentration condition i for each phenotypic parameter ψ.

Following this operation, a series of distances for the biological samples is computed for every pair made of a control κ and a biological sample in the series of concentration (S₁, S₂, . . . , Sᵢ).

$${d}_{κ , j}^{( ψ )} = D ( {C}_{κ}^{( ψ )} , {S}_{i}^{( ψ )} ) , i = 1 … I , κ = 1 … K ,$$

where distance function D can be a Quadratic Form (QF) distance, a Wasserstein distance, Sinkhorn distance, a quadratic-χ² distance or any other distance operating on numerical vectors representing distributions, probability mass functions, histograms, or other representations of relative likelihood.

A vector denoted a_{[κ,ψ]} is a vector which contains a series of distances d_{κ,i}^{(ψ)} measured for biological parameters ψ and a control condition κ, at concentrations i=1 . . . I,

$${a}_{[ κ , ψ ]} = [ \begin{matrix}{d}_{κ , 1}^{( ψ )} = D ( {C}_{κ}^{( ψ )} , {S}_{1}^{( ψ )} ) \\ {d}_{κ , 2}^{( ψ )} = D ( {C}_{κ}^{( ψ )} , {S}_{2}^{( ψ )} ) \\ ⋮ \\ {d}_{κ , I}^{( ψ )} = D ( {C}_{κ}^{( ψ )} , {S}_{I}^{( ψ )} )\end{matrix} ]$$

The measurements representing multiple biological parameters ψₚ where p=1 . . . P, can be grouped into a 2-dimensional array (i.e., a two-way tensor):

$${a}_{[ κ ]} = [ [ \begin{matrix}{d}_{κ , 1}^{( {ψ}_{1} )} = D ( {C}_{κ}^{( {ψ}_{1} )} , {S}_{1}^{( {ψ}_{1} )} ) \\ {d}_{κ , 2}^{( {ψ}_{1} )} = D ( {C}_{κ}^{( {ψ}_{1} )} , {S}_{2}^{( {ψ}_{1} )} ) \\ ⋮ \\ {d}_{κ , I}^{( {ψ}_{1} )} = D ( {C}_{κ}^{( {ψ}_{1} )} , {S}_{I}^{( {ψ}_{1} )} )\end{matrix} ] [ \begin{matrix}{d}_{κ , 1}^{( {ψ}_{2} )} = D ( {C}_{κ}^{( {ψ}_{2} )} , {S}_{1}^{( {ψ}_{2} )} ) \\ {d}_{κ , 2}^{( {ψ}_{2} )} = D ( {C}_{κ}^{( {ψ}_{2} )} , {S}_{2}^{( {ψ}_{2} )} ) \\ ⋮ \\ {d}_{κ , I}^{( {ψ}_{2} )} = D ( {C}_{κ}^{( {ψ}_{2} )} , {S}_{I}^{( {ψ}_{2} )} )\end{matrix} ] , … , [ \begin{matrix}{d}_{κ , 1}^{( {ψ}_{P} )} = D ( {C}_{κ}^{( {ψ}_{P} )} , {S}_{1}^{( {ψ}_{P} )} ) \\ {d}_{κ , 2}^{( {ψ}_{P} )} = D ( {C}_{κ}^{( {ψ}_{P} )} , {S}_{2}^{( {ψ}_{P} )} ) \\ ⋮ \\ {d}_{κ , I}^{( {ψ}_{P} )} = D ( {C}_{κ}^{( {ψ}_{P} )} , {S}_{I}^{( {ψ}_{P} )} )\end{matrix} ] ]$$

These arrays can be further grouped into a tensor A [symbol] storing distance values d_{κ,i}^{(ψ)} for each phenotypic parameter ψ, each concentration i, and each condition κ and written in a simpler notation as:

$$A = [ \begin{matrix}{d}_{1 , 1}^{( {ψ}_{1} )} & {d}_{1 , 1}^{( {ψ}_{2} )} & … & {d}_{1 , 1}^{( {ψ}_{P} )} & {d}_{2 , 1}^{( {ψ}_{1} )} & {d}_{2 , 1}^{( {ψ}_{2} )} & … & {d}_{2 , 1}^{( {ψ}_{P} )} \\ {d}_{1 , 2}^{( {ψ}_{1} )} & {d}_{1 , 2}^{( {ψ}_{2} )} & … & {d}_{1 , 2}^{( {ψ}_{P} )} & {d}_{2 , 2}^{( {ψ}_{1} )} & {d}_{2 , 2}^{( {ψ}_{2} )} & … & {d}_{2 , 2}^{( {ψ}_{P} )} \\ ⋮ & ⋮ & ⋱ & ⋮ & ⋮ & ⋮ & ⋱ & ⋮ \\ {d}_{1 , I}^{( {ψ}_{1} )} & {d}_{1 , I}^{( {ψ}_{2} )} & … & {d}_{1 , I}^{( {ψ}_{P} )} & {d}_{2 , I}^{( {ψ}_{1} )} & {d}_{2 , I}^{( {ψ}_{2} )} & … & {d}_{2 , I}^{( {ψ}_{P} )}\end{matrix} , … , \begin{matrix}{d}_{K , 1}^{( {ψ}_{1} )} & {d}_{K , 1}^{( {ψ}_{2} )} & … & {d}_{K , 1}^{( {ψ}_{P} )} \\ {d}_{K , 2}^{( {ψ}_{1} )} & {d}_{K , 2}^{( {ψ}_{2} )} & … & {d}_{K , 2}^{( {ψ}_{P} )} \\ ⋮ & ⋮ & ⋱ & ⋮ \\ {d}_{K , I}^{( {ψ}_{1} )} & {d}_{K , I}^{( {ψ}_{2} )} & … & {d}_{K , I}^{( {ψ}_{P} )}\end{matrix} ]$$

### Tensor Feature Extraction

A tensor A obtained from a series of measurements forms a unique compound fingerprint, as it contains all the phenotypic characteristics of a tested compound. This tensor A can be “simplified” using tensor feature extraction techniques. The disclosed methods take advantage the fact that each of the vectors (a tensor fibers) is physically associated with changes in cellular responses across the I concentrations of a test compound. Therefore, rather than being disconnected, independent values, the calculated distribution distances in the tensor fibers form a dose-response curve. Thus, the tensor A can be simplified by reducing or compressing the information stored in these response curves.

To simplify or compress the data contained within tensor A, disclosed methods use the distribution distances d with each of the tensor fibers a to identify features representing the drug-response at a concentration I. One such technique includes determining, for each tensor fiber a, a range between the values of the distance distributions contained therein, and a maximum rate of change between those distance distributions. The distances d may be plotted against the concentration levels for a tensor fiber a for a phenotypic parameter ψ. The difference between the maximum and minimum distribution distance may be the range. The maximum rate of change may be represented by the steepest point on the curve.

Therefore, the full tensor representation can be simplified by calculating, for each fiber a_{[κ,ψ]} of the tensor A, a range α between distances 1 to I and a maximum rate of change 8 between distances from 1 to I-1:

$${α}_{κ}^{( ψ )} = range ( {d}_{κ , 1}^{( ψ )} , {d}_{κ , 2}^{( ψ )} , … , {d}_{κ , I}^{( ψ )} ) , κ = 1 … K$$

$${β}_{κ}^{( ψ )} = {max arg}_{i} ( \frac{{d}_{κ , i}^{( ψ )} - {d}_{κ , i + 1}^{( ψ )}}{g ( {S}_{i + 1} ) - g ( {S}_{i} )} ) , κ = 1 … K , i = 1 … I - 1$$

The range and maximum rate of change may be “extracted” from the tensor A by calculating these values for each tensor fiber a and adding them as entries to a single two dimensional response curve feature vector. The optional function g(.) provides a transformation ensuring the linearity of the concentration range (e.g. g(x)=log₁₀(x)).

After applying feature extraction techniques, the tensor A is reduced to a smaller tensor R.

$$R = [ \begin{matrix}{α}_{1}^{( {ψ}_{1} )} & {α}_{1}^{( {ψ}_{2} )} & … & {α}_{1}^{( {ψ}_{P} )} & {α}_{2}^{( {ψ}_{1} )} & {α}_{2}^{( {ψ}_{2} )} & … & {α}_{2}^{( {ψ}_{P} )} \\ {β}_{1}^{( {ψ}_{1} )} & {β}_{1}^{( {ψ}_{2} )} & … & {β}_{1}^{( {ψ}_{P} )} & {β}_{2}^{( {ψ}_{1} )} & {β}_{2}^{( {ψ}_{2} )} & … & {β}_{2}^{( {ψ}_{P} )}\end{matrix} , … , \begin{matrix}{α}_{K}^{( {ψ}_{1} )} & {α}_{K}^{( {ψ}_{2} )} & … & {α}_{K}^{( {ψ}_{P} )} \\ {β}_{K}^{( {ψ}_{1} )} & {β}_{K}^{( {ψ}_{2} )} & … & {β}_{K}^{( {ψ}_{P} )}\end{matrix} ]$$

Consequently, there is a resource savings in space required for storing the associated data. The tensor R can be further vectorized, and the resultant vector r may be used as input for a machine-learning based toxicity classification model. For the simplest case where K=1 (there is only one control measurement κ, e.g., a negative control), the r vector takes form:

$$r = {[ \begin{matrix}{α}_{1}^{( {ψ}_{1} )} & {α}_{1}^{( {ψ}_{2} )} & … & {α}_{1}^{( {ψ}_{P} )} & {β}_{1}^{( {ψ}_{1} )} & {β}_{1}^{( {ψ}_{2} )} & … & {β}_{1}^{( {ψ}_{P} )}\end{matrix} ]}^{T}$$

Another feature extraction technique that may be employed with the present embodiments, is the computation of parameters associated with the parametric sigmoidal representation of these curves. For instance, with a 3-parameter log-logistic model for the dose-response curves feature extraction may include capturing the values associated with asymptotes and the inflection point of the curve.

In various embodiments, the disclosed methods can be implemented using two-, three-, and higher dimensional versions of the probability mass function approximation. This modification may be especially relevant for cases in which there is a significant association or dependence between two or more biological or biophysical parameters. In this setting, instead of computing distances/dissimilarities between 1-D representations of D formed by data obtained by each of the biological/biophysical parameters, distances may be calculated between approximations of 2-D (or n-D, in general) D functions formed by several biophysical/biological parameters. For instance, the distances in 2-D can computed using biological parameters ψ₁ and ψ₂:

$${d}_{κ , i}^{( {ψ}_{1} , {ψ}_{2} )} = D ( {C}_{κ}^{( {ψ}_{1} , {ψ}_{2} )} , {S}_{i}^{( {ψ}_{1} , {ψ}_{2} )} ) , i = 1 … I , κ = 1 … K$$

Regardless of the distance function choice, or the dimensionality, the final feature vectors quantitatively represent the cellular stress phenotype caused by a test agent. What remains is to classify the response curve feature vectors r.

### Automated Gating

An embodiment provides for the use of model driven automatic gating (although, the use of gating algorithms is optional). Herein, state-of-art techniques of mixture modeling with or without proprietary additions may be added to the algorithm. The system may rely on an iterative approach to improve efficiency of the assay.

In an embodiment, the gating technique comprises 3 skew-normal probability distributions representing “live cells,” “dying cells,” and “dead cells” (debris). Depending on the data, an existing (e.g., old validated) model may be used or a new generated based on the controls. For example, it is possible to proceed by calculating the total log-likelihood (LL) for each mixture model. Specific models for which LL is higher are then retained for future use.

### Response Curve Classification

Embodiments provide classification methods, wherein subsequent analyses are performed using machine learning techniques. These techniques may analyze and classify a response curve feature vectors computed to each analyzed agent to produce a probability that an associated agent demonstrates a toxicity characteristic at one or more concentration levels I.

Embodiments provide a toxicity classifier model that uses a logistic regression model regularized by an elastic net. This logistic model is multidimensional meaning that it includes multiple regressions, as it must simultaneously utilize information from each of the flow cytometry detection parameters encoded in the response curve feature vector r. The toxicity classifier model is trained by repeated cross-validation and grid search for B and the values controlling the LASSO and ridge penalties (λ₁ and λ₂). The optimally fit model then becomes the toxicity classifier model, allowing calculation of the likelihood that a response curve feature vector, or any of its columns, can be assigned to the “yes,” e.g., high cell-stress class. A final risk score, or Cell Health Index (CHI), may be the probability with which the test agent's response curve feature vector, or its columns, can be assigned to the “yes” class according to the boundary between the classes described by the toxicity classifier model.

Furthermore, embodiments may improve the accuracy of the final risk score through independent validation. A series of unidimensional classifiers, simple regressors, may be trained and applied to the phenotypic parameters separately, calculating the probability of “yes” class assignment if only data for each phenotypic parameter were considered in isolation. These single parameter classifications may produce an additional “fingerprint” of scores that can be interpreted as indicating the relative ability of each parameter to form a prediction aligned with the final score (i.e., CHI). This information may indicate the biological relevance of an individual phenotypic parameter. But, the predictive value of individual phenotypic parameters cannot be assumed a priori to be equal. Moreover, the elastic net regressor can provide a ranking of features based on their contribution to the trained toxicity classifier model. This ranking provides information about a phenotypic predictors' “quality” and relevance in a statistical sense.

Embodiments provide for the determination of a risk score based in proximity of a classified response curve feature vector, or tis columns, to a boundary lying between two or more risk classes. In a two-dimensional, or binary, setting, the response curve feature vector may be classified and attributed to a point or location within a 2-D space, in which, two classes of risk are delineated. In an example, the further the point is from a boundary between the risk classes, the higher the associated probability that the phenotypic parameter at issue, belongs within the risk class to which it was classified. A response feature vector column assigned to a “yes” risk class and laying far from the boundary between risk classes may be considered to have a high probability of risk and thus may receive a high CHI. This CHI may represent a prediction of the likelihood that a compound has high toxicity risk. This “high toxicity risk” may translate to a drug candidate failing because of safety concerns (poor animal trial performance, severe side effects in human clinical trials, withdrawal from the market, etc.) or an industrial/agricultural chemical causing safety problems through human exposure.

The risk score, i.e. CHI, may be used as a threshold for screening selection of agent concentrations in future rounds of agent testing. Agents and concentrations lying below a threshold risk score may be discarded from future rounds of testing. Alternatively, agents or concentrations lying above a risk score threshold may be discarded and removed from future testing populations. In this way, the classification techniques provide risk cores that may be used in agent testing population screening. This may reduce the amount of duplicative or unnecessary testing performed on cells that are not at suitable risk for developing toxicity characteristics after exposure to an agent or concentration.

The above embodiments are disclosed with reference to an implementation including elastic net regression but, it is not the only classifier suitable for delivering the expected results. Other embodiments include the use of classifiers such as support vector machines (SVM), neural networks (NN), or Bayesian approaches.

It should be noted that the binary problem formulation is not the only framework in which the disclosed embodiments may be executed. As discussed herein, one can design a number of controls reflecting several feasible phenotypes. Each of these phenotypes may be associated with a class γ, leading to a multiclass classification problem utilizing (Γ−1)-logits

$$log \frac{P r ( {y}_{i} = γ \vert x )}{P r ( {y}_{i} = Γ \vert x )} = X {B}_{k} for γ = 1 , 2 , … , Γ - 1.$$

Such embodiments may be implemented using multinomial regression with the multiclass elastic net penalty or another multiclass classification method.

### Classification Model Training

In order to obtain a high degree of accuracy in classification of phenotypic parameters of a response curve feature vector as being high or low risk, it is important that the toxicity classifier model be trained. Training provides example instances of the known outcome classes among which the toxicity classifier model is intended to discriminate.

Training the toxicity classifier model may include use of a training set including both: agents with a known risk class, such as drugs with known safety histories indicating either high or low toxicity risk; and 2) descriptive data in the same feature space that the classifier will use to estimate outcome probability such as, cellular phenotypic data associated with agent exposure. These data sets may be used tune the classifier. Tuning, or optimizing the classifier enables it to predict risk class assignment probability from inputs based on phenotypic parameters of cells exposed to a test agent.

Embodiments provide for the generation of a training set by assembled 300 or more known agents drawn from on-market pharmaceuticals, withdrawn drugs, research compounds, and industrial/agricultural compounds. These agents may be assigned to one of two historically known outcome classes: the “yes” class or “positive” class representing known toxicity and associated high expectation of acute cell stress) and the “no” class, i.e. “negative” class. Classification may be based on curated information gathered from the scientific literature, clinical trial results, and/or known commercial histories. For many compounds that have known toxic side effects, scientific research literature directly documents cellular effects, e.g., mitochondrial dysfunction, reactive oxygen species generation, etc. These agents serve as perfect training instances for the high risk class. For examples of low risk class agents, agent development history data in classification may be used, such as clinical trials, or its commercial history after going on-market, etc. Agents with no reported history of cytotoxicity during development may be assigned to the low risk class.

Once all risk class assignments have been made, all 300 or more agents may be physically processed through the Cell Health Screen to produce response curve feature vectors. Every agent in the training set may then have two associated indicators: the binary assignment to the historically known outcome (“ground truth”); and the empirical measurement of cellular stress phenotype. Visualized in a feature space, the two risk classes may form clouds containing the phenotypic parameter features. If the two clouds do not overlap except as needed to form a boundary, then the classifier model may be sufficiently trained to be able to accurately predict future risk class assignment of response curve feature vectors.

Embodiments provide for training the toxicity classifier model for one dimension or one phenotypic parameter. This may include training for all the feature values for that phenotypic parameter from all 300 or more training agents as applied to one logistic regression. A logistic model may be optimized by finding parameters for a curve that most effectively separates the populations of feature values from the “yes” and “no” risk classes. For a multidimensional model, this process may be performed computationally for all phenotypic parameters simultaneously, resulting in a model that includes the most parsimonious separation of the “yes” and “no” training set vectors along all measurement axes.

Further, the model may be regularized to minimize the potential detrimental influences of a large number of predictors (i.e. measurement features used as input). These possible detrimental effects include: predictive signals that are unevenly distributed among input features; and predictors that are correlated and thus not entirely independent. In elastic net regularization, two types of model penalties are implemented: L₁ (LASSO regression) and L₂ (Ridge regression). These regularizations penalize the size of parameter estimates in order to completely eliminate some of them (LASSO) or shrink them continuously towards zero (Ridge). Specifically, LASSO techniques penalize the sum of their absolute values (L₁ penalty), and Ridge regression penalizes the sum of squared coefficients (L₂ penalty). An advantage of the elastic net techniques is that they combine the L₁ penalty, which is suitable for a situation in which only a few predictors actually meaningfully predict response; and, the L₂ penalty, which is advantageous when multiple predictors providing similar predictive value.

Embodiments provide for a classifier model that is formulated as a binary decision with two class-conditional probabilities:

$$log \frac{P r ( {y}_{i} = + 1 \vert x )}{P r ( {y}_{i} = - 1 \vert x )} = XB where ( - 1 , + 1 ) are class labels .$$

The use of elastic net regularization leads to the model:

$$\hat{B} = arg {min}_{B} \{ \frac{1}{2 n} \vert \vert Y - XB \vert {\vert}_{2}^{2} + {λ}_{2} {\vert \vert B \vert \vert}_{2}^{2} + {A}_{1} \vert \vert B \vert {\vert}_{1} \} .$$

It should be noted that, the disclosed embodiments are designed to predict toxicity risk arising from cellular energy metabolism, ion flux, reactive radical formation, and similar mechanisms that cause acute cellular stress rapidly via physiological phenomena that are detectable with commercially available fluorescent dyes. Other types of chemical safety problems, such as teratogenic effects or hormonal disruption, cannot be detected by our physical screen design. This design choice was driven by the fact that cellular effects, such as mitochondrial dysfunction and ion imbalances, are known to underlie several more common adverse safety events such as liver damage, cardiac dysfunction, and neuropathies. Teratogenic effects and hormonal disruption are problems that arise more often in the context of pregnancy, child development, or cancer potentiation; as such, these are also important risks to detect, but they need to be addressed by a separate design process. Consequently, the disclosed training techniques are implemented with training data that may be curated to avoid inadvertently training the classifier with outcome types that cannot be informed by the disclosed screen's measurement parameters.

### Cell Cycle

Embodiments herein described allow measurements of coordinated protein (or other marker) expression in populations of cells as a function of cell cycle (e.g. G1, S, G2M), and to determine cell-cycle-dependent effects of the test compounds. Multi-parametric analysis may thus be conducted by analyzing the effect of each perturbant at different concentrations and/or time points to investigate the effect of said compounds on the various cellular parameters (e.g., mitochondrial membrane potential, nuclear or cytoplasmic membrane permeability, ROS, cell death or apoptosis).

An example of cell-cycle dependent analysis is based on the measurement of Cyclin A2 expression in normal (unperturbed) cells. Herein, the possible “states” include Cyclin A2 negative, Cyclin A2 low and Cyclin A2 high. Similarly, for phospho-histone 3 (P-H3), which is a second marker in cell-cycle analysis, the possible “states” include “negative” and “positive”. These two cell-cycle markers may also be analyzed in combination, thus yielding nine different possible combinations (“states”). It is not always necessary to investigate all possible “states” because all the states may not exist in normal biological space (sparse matrix).

Accordingly, depending on the cell cycle state a particular cell is in, differential perturbations caused by drugs or compounds of interest can be investigated by populating cells in discrete (normal) matrix elements. As an example, drugs which block normal progression from mitosis back into G1, which cause quantitative changes in “normal” matrix populations (i.e., accumulation of cells into “late” (normal) cell cycle compartments (e.g. G2 and M)) and/or deplete cells in the G1 phase, can be analyzed in concert using Cyclin A2 and/or P-H3 staining. Similarly, a drug which prevents separation of daughter nuclei would be expected to show a different quantitative fingerprint pattern compared to a drug which arrests cells in S-phase (e.g. a drug which inhibits new DNA synthesis). Accordingly, compounds which cause cells to appear in different matrix elements not only creates a unique signature, but also the specific matrix element that is occupied could provide information regarding the mechanism of drug action. For example, expression of Cyclin A2 in G1 and or M can be the result of a proteasome inhibitor preventing normal Cyclin A2 degradation.

### Multiple Cell Type Assay Systems

In an embodiment, the present invention provides for methods for assaying cellular states using a plurality of cell types, e.g., two or more cell lines (from tissue culture) in a single assay. One advantage of this approach is it allows analyses of DNA damage/responses. An additional advantage is that it allows studies of both constitutive and inducible signaling pathways in the same assay (using one cell line with constitutive expression and another that can activate the same pathway using an appropriate agonist). Using two (or more) cell lines simultaneously, it will be possible to cover multiple signaling pathways in one assay.

For example, using human myeloid cell lines (derived from patients with myeloid leukemia), one cell line responsive to LPS will activate NF-κB and PI3 Kinase pathways, while another responsive to TNF-α will activate multiple MAP kinase pathways; in both cases, upstream (Iκ kinase for NF-κB) and downstream (P-S6 for ERK and mTOR for PI3K) can be evaluated. In addition, these assays can include DNA damage/response markers, as indicated above. The responding cell line in cell mixtures can be identified using either DNA content (some cell lines are diploid; others are aneuploid with different abnormal DNA content), or biological characteristics (cell surface markers), or cells can be “barcoded” (G. Nolan et al.). Finally, signaling assays can include cell cycle analysis (e.g. DNA content) to allow correlation of signal transduction pathway responses with cell physiology in response to the same drugs.

From careful consideration of the foregoing description in light of the references cited herein, one skilled in the art can ascertain the characteristics of inventions and embodiments herein described and will be enabled thereby to undertake a wide variety of changes and modifications thereof without departing from the spirit and scope thereof.

All publications and patents cited herein are incorporated herein by reference in their entireties, particularly in the parts most pertinent to the discussion thereof.

### EXAMPLES

The following examples are provided by way of illustration and are in no way exhaustive, exclusive or limitative of other aspects and embodiments of inventions herein described.

### Example 1: Assessing Cytotoxicity Risk of Excipient Compounds

### 1. INTRODUCTION

Example embodiments of the invention are processes for detecting changes in cellular biological state. Such changes may result from any perturbation that causes a measurable effect relative to a control, which can be detected by an optical signature on a cytometry platform, such as flow cytometry (FC). Here we describe a specific example reduced to practice, where it takes the form of an acute cell stress screen performed on an automated FC platform. One practical application is the assessment of potential human safety risks from chemical compound exposure for either candidate pharmaceuticals or new industrial/agricultural compounds. Early pre-clinical pharmaceutical development and safety assessment of industrial/agricultural compounds will both benefit from new processes that reduce cost, increase efficiency of test material use, and increase predictive power for safety risk, relative to the current industry practices that rely upon extensive animal trials. In the pharmaceutical industry, other types of automated biological screen have been tested as potential tools for improving pre-clinical toxicology assessment (Bowes et al., 2012; Pottel et al., 2020; Whitebread et al., 2005); however, these applied screens are commonly unidimensional, with low information content, requiring multiple separate workflows to assemble adequate multidimensional information. Consequently, they can be relatively expensive and labor-intensive for eliminating candidate chemical structures associated with general cell health issues. The below example demonstrates production of multidimensional cellular phenotypic data that can subsequently be converted to predictive estimates of human toxicity risk for individual chemical compounds.

In the study described here, a specific embodiment, in the form of an acute cell stress screen called the Cell Health Screen, was used to estimate toxicity risk for 40 excipient compounds. Excipients serve as vehicles, preservatives, solubilizers, and colorants for drugs, food, and cosmetics. They are considered to be inert at biological targets; however, several reports suggest that some could interact with human targets and cause unwanted effects (Bora et al., 2019; Burbacher et al., 2005; Chevalier et al., 2015; Ivanovska et al., 2014; Pifferi & Restani, 2003; Rowe & Rowe, 1994; Walsh et al., 2018; Yang et al., 2018). Sec Table 1 for the complete list of all 40 excipients used in this study, including their application types.

The purpose of this study was to assess the toxicity risk estimation provided by the Cell Health Screen relative to information from panels of in vitro pharmacology assays that were also designed to detect toxicity risk during pharmaceutical development. This study was performed with outside collaborators who have expertise in the use of the in vitro pharmacology assays. These in vitro assay panels detect whether chemical compounds directly interact with biomolecular targets known to be associated with toxic side effects in humans (mostly enzymes, cell surface receptors, and other proteins that participate in signaling pathways) (Pottel et al., 2020). For these in vitro assay panels, assessment of toxicity risk is an interpretation of how “promiscuous” a compound is (how many different biomolecular targets it engages) and whether or not it potently engages certain toxicity-associated targets at low concentrations. As such, the interpretation process is somewhat subjective. In contrast, the Cell Health Screen uses a feature extraction and ML classifier strategy described above, to reduce all cellular phenotypic changes caused by a chemical compound to a single probability value, from 0 to 1. This is a quantitative toxicity risk estimation relative to a training set of compounds used to train the ML classifier. Therefore, comparing results from the Cell Health Screen to the in vitro pharmacology panels is a matter of comparing the trend in ML classifier probability values, across all 40 excipients, with their relative degrees of promiscuity and target interaction potency observed in the in vitro pharmacology panels.

### 2. METHODS AND MATERIALS FOR THE ASEDASCIENCES SYSTEMETRIC CELL HEALTH SCREEN

### 2.1 Source of Test Compounds

All 40 excipients were provided from the Novartis compound library after QC analysis confirmed >99% purity. All excipients were dissolved in DMSO and provided as 10 mM stocks. In choosing candidate compounds for the study, we considered limitations that eliminated some excipients from our list, such as low solubility, aggregation, color quenching, and chemical stability.

TABLE 1
Selected excipients and their application in drugs and/or foodstuff

| Excipient | CASRN | Application |
|---|---|---|
| Gentisic acid | 490-79-9 | Antioxidant |
| (2,5-dihydroxybenzoic acid) | | |
| Propylene glycol | 57-55-6 | Binder/solubilizer |
| Hydroxypropyl cellulose | 9004-64-2 | Binder/solubilizer |
| Methyl cellulose | 9004-67-5 | Binder/solubilizer |
| PEG 400 | 25322-68-3 | Binder/solubilizer |
| Polysorbate 80 | 9005-65-6 | Binder/solubilizer |
| Succinic acid | 110-15-6 | Binder/solubilizer |
| Hydroxylpropyl methyl cellulose | 9004-65-3 | Coating |
| Oleic acid | 112-80-1 | Coating |
| Triethyl citrate | 77-93-0 | Coating |
| D&C Red #28 (Phloxine B) | 18472-87-2 | Colorant |
| FD&C Red #3 (Erythrosine | 16423-68-0 | Colorant |
| extra bluish) | | |
| Methylene blue hydrate | 67183-68-0 | Colorant |
| Tartrazine (FD&C Yellow #5) | 1934-21-0 | Colorant |
| D-Mannitol | 69-65-8 | Compacting agent |
| Lactose | 63-42-3 | Compacting agent |
| 1-(o-Tolyl)biguanide | 93-69-6 | Detergent |
| Butyl paraben | 94-26-8 | Disintegrant |
| Povidone (PVP or | 9003-39-8 | Disintegrant |
| Polyvinylpyrrolidone) | | |
| Aspartame | 22839-47-0 | Flavoring |
| Glycerine | 56-81-5 | Flavoring |
| Saccharin | 81-07-2 | Flavoring |
| Diethyl phthalate | 84-66-2 | Lubricant |
| Stearic acid | 57-11-4 | Lubricant |
| Palmitic acid | 57-10-3 | Lubricant/food additive |
| Thymol | 89-83-8 | Preservative |
| (2-isopropyl-5-methylphenol) | | |
| Chloroxylenol (4-chloro- | 88-04-0 | Preservative |
| 3,5-dimethylphenol) | | |
| Benzethonium chloride | 121-54-0 | Preservative |
| Benzoic acid | 65-85-0 | Preservative |
| Benzyl alcohol | 100-51-6 | Preservative |
| Cetylpyridinium chloride | 6004-24-6 | Preservative |
| monohydrate | | |
| Ethylparaben | 120-47-8 | Preservative |
| Methylparaben (methyl | 99-76-3 | Preservative |
| 4-hydroxybenzoate) | | |
| Phenylmercuric acetate | 62-38-4 | Preservative |
| Propyl gallate (E310) | 121-79-9 | Preservative |
| Propylparaben | 94-13-3 | Preservative |
| Thimerosal | 54-64-8 | Preservative |
| Phenol | 108-95-2 | Preservative/antiseptic |
| Ethylene diamine | 107-15-3 | Solubilizer |
| Sodium dodecyl sulfate | 151-21-3 | Surfactant |

### 2.2 Detailed description of the Cell Health Screen and its Execution

### 2.2.1 Overview of Screen Design

The Cell Health Screen is a multiparametric acute cell stress assay, using a panel of fluorescent physiological reporting dyes, on an automated flow cytometry platform. Rather than simply producing dose-response curves for all individual biological readouts, features are generated by computing custom-defined distance functions between test and control wells. All test compounds are represented as feature vectors, after which the analysis algorithm employs a logistic regression model to classify test compounds relative to a training set. This machine learning (ML) approach integrates all measured readouts into a single predictive statistical model. This data processing strategy has two notable advantages: 1) feature extraction and data reduction avoid subjective gating of flow cytometry data; 2) the ML classifier has been trained with 300 known compounds comprised of on-market and withdrawn drugs and research compounds. This training set empirically covers the full range of possible phenotypes in the Cell Health Screen, from no-response to acute stress, with sufficient representation across the spectrum. Training set compounds were assigned to binary classes (“yes”=expectation of high cell stress or “positive” phenotype; “no”=no expectation of positive phenotype). This externally established ground-truth was based upon manually curated information from research literature and, where applicable, clinical trial results and commercial/regulatory histories.

For an unknown test compound, the ML classifier uses all the FC parameter features describing compound response, simultaneously, to predict the final assignment. This is achieved by calculating the probability of assigning that compound's screen phenotype to the “yes” class defined by the training set. By specifying the problem as a classification challenge, the data analysis pipeline assures that any apparent lack of coordinated change among biological readouts presents no interpretation challenge. All phenotypic data are treated simply as input features to a statistical model. In contrast, many conventional flow cytometry assays require strict mechanistic interpretation of every measured biological readout. often resulting in conflicting conclusions (e.g. if reactive oxygen species increase, but glutathione is unaffected, which should be “believed”?). The final probability score, or Cell Health Index, is a quantitative assessment of a multiparametric phenotype's similarity to a diverse set of known good and bad actors. Finally, choosing HL60 as our reporter cell line means that the screen is explicitly designed not to detect instances in which a parent compound only causes cellular toxicity via metabolites. This design feature provides certain advantages, exemplified by the fact that our screen reports a stark difference between terfenadine (highly cytotoxic when not metabolized) and its metabolite fexofenadine.

### 2.2.2 Physical Execution Summary

In a 384-well platform. HL60 cells are exposed to a 10-step, 3× dilution series of each test compound (5 nM-100 μM) for 4 hours at 37° C. with 5% CO₂. Each dilution series is screened in duplicate, occupying a total of 20 wells, allowing 16 test compounds to be assayed on each plate. Each row contains one positive and one negative control well, for a total of 16 matched control pairs on each assay plate. Compound formatting, cell deposition, and dye application are performed robotically, so that final assay conditions comprise 100,000 cells in a 40 μl volume. After compound exposure, live cells are rapidly stained with a panel of fluorescent dyes that report physiological signatures of both mitochondrial dysfunction and gross cell stress. Fluorescence data are collected using automated flow cytometry with no gating. In addition, forward scatter and side scatter at 488 nm are acquired for conversion into a cell morphology parameter. Well-specific flow cytometry data files, with an accompanying map of well contents, are moved to cloud infrastructure where the automated algorithm for quality control and ML classification is triggered.

### 2.2.3 HL60 Cell Culture Production

HL60 cells are produced as suspension cultures in non-treated 850 cm² roller bottles with vented caps, at 1 RPM, 5% CO₂, and 37° C. Culture medium is RPMI 1640 without glucose, supplemented with 10 mM galactose and 10% dialyzed heat-inactivated FBS. Further supplementation follows ATCC standard recommendations for this cell line. Culture density is maintained at or below 1×10⁶ cells/ml. A new production lineage of HL60 cells is started each month, and a crossover screen is performed in which the old and new production lineages are compared by using a set of 16 reference compounds to produce a known set of stress phenotypes. In this way, variation of screen performance is minimized by producing all screening cell populations within a narrow range of passage numbers, each checked for consistency of phenotypic performance with reference compounds.

### 2.2.4 Test Compound Formatting, Cell Exposure, and Staining

Test compounds are screened in sets of 16. Each set is formatted in two replicate 384-well plates (Eppendorf Protein LoBind®, catalog number 951040589) for assays with two subsets of fluorescent dyes. (Spectral overlap and DMSO limitation prevent simultaneous use of the complete dye panel.) Compounds in these replicate plates are identical except for positive controls, which have been chosen to produce an optimal response within each subset of fluorescent reporter dyes. Test compound dilution series and controls are formatted on a Biomek® 4000. Each compound is formatted as a 10-step, 3× dilution series, in duplicate, on each of the two plates. Negative control wells contain the diluent used for both the test compound dilution series and positive controls. Both positive and negative controls are distributed to plate wells from a single initial reservoir of each control mixture. Final assay concentration range for test compounds is 5 nM to 100 μM. The diluent is RPMI 1640 (supplemented as above) with final working concentration of DMSO normalized to 1% in all wells. Prior to cell deposition, assay plates containing formatted compounds are sealed and stored at room temperature, protected from light, for 2 hours, to allow binding equilibrium between serum components and test compounds. A Biomek N×P is used to deposit cells in all wells, at a density of 2.5×10⁶ cells/ml, in a final assay volume of 40 μl per well (approximately 100,000 cells per well). After cell deposition, each assay plate is sealed with breathable plate sealer, shaken at 2,200 RPM for 10 seconds (Illumina® High-speed microplate shaker), and incubated for 4 hours at 37° C. with 5% CO₂.

### 2.2.4.1 First Fluorescent Dye Mix and Staining Conditions

Dye mix buffer is 1×PBS with 4% FBS, filter sterilized. The dye set consists of: Calcein AM, SYTOX™ Red, MitoSOX™ Red, and Monobromobimane (Life Technologies catalog numbers C1430, S34859, M36008, and M20381, respectively). Dye concentrations were previously optimized to produce maximum dynamic range between positive and negative control wells. Prior to deposition of dye mix, the assay plate is removed from its 4 hour incubation, and cells are gently pelleted at 300×g for 2 minutes. A Biomek NX^{P} is then used to aspirate 20 μl of each well volume, after which 20 μl of dye mix is deposited in all wells. After dye deposition, the plate is re-sealed with its breathable plate sealer, shaken 2× at 2,200 RPM for 5 seconds each time (1 second interval), and incubated for 10 minutes at 37° C. with 5% CO₂. The plate is then rapidly cooled to room temperature for 1 minute in a shallow water bath, after which acquisition of flow cytometry data is started immediately.

### 2.2.4.2 Second Fluorescent Dye Mix and Staining Conditions

Dye mix buffer is 1×PBS with 4% FBS, filter sterilized. The dye set consists of: JC-9, propidium iodide, and Vybrant® DyeCycle™ Violet (Life Technologies catalog numbers D22421, P3566, V35003, respectively). Dye concentrations were previously optimized to produce maximum dynamic range between positive and negative control wells. Cell pelleting and dye deposition are performed as above, in 2.2.4.1. After dye deposition, the plate is re-sealed with its breathable plate sealer, shaken 2× at 2,200 RPM for 5 seconds each time (1 second interval), and incubated for 30 minutes at 37° C. with 5% CO₂. The plate is then allowed to sit at room temperature for 15 minutes, protected from light. Acquisition of flow cytometry data is started immediately after this 15 minute period.

### 2.2.5 Acquisition of Flow Cytometry Data

Flow cytometry data are acquired with a CyAn™ ADP flow cytometer (Beckman Coulter) with automated sampling performed by a HyperCyt® autosampler (Intellicyt). Autosampler settings are optimized to aspirate ≥10,000 cells per well. As described in Section 2.2.4 above, the complete set of fluorescent dyes is applied as two non-overlapping mixtures on replicate assay plates. Therefore, two separate flow cytometer acquisition protocols are used. Note that all channels are acquired with no gating. Triggering is on Forward Scatter with Threshold=5%. Acquisition channel settings in Summit (version 4.3) for these two protocols are described in Table 2 and Table 3.

TABLE 2
Summit 4.3 acquisition settings for the
first dye mix in the Cell Health Screen

| | CyAn ADP | | | |
|---|---|---|---|---|
| Dye | Channel Name | Peak/Area/Log | Voltage | Gain |
| NA | Forward Scatter | Peak/Area | NA | 2.5 |
| NA | Side Scatter | Peak/Area | 533 | 1.0 |
| Calcein AM | FITC | Log | 520 | 1.0 |
| MitoSOX Red | PE-Texas Red | Log | 750 | 1.0 |
| monobromobimane | Violet 2 | Log | 385 | 1.0 |
| SYTOX Red | APC | Log | 675 | 1.0 |

TABLE 3
Summit 4.3 acquisition settings for the
second dye mix in the Cell Health Screen

| | CyAn ADP | | | |
|---|---|---|---|---|
| Dye | Channel Name | Peak/Area/Log | Voltage | Gain |
| NA | Forward Scatter | Peak/Area | NA | 2.5 |
| NA | Side Scatter | Peak/Area | 533 | 1.0 |
| JC-9 (green | FITC | Log | 335 | 1.0 |
| emission) | | | | |
| JC-9 (red emission) | PE-Texas Red | Log | 525 | 1.0 |
| Propidium iodide | PE-Cy7 | Peak/Area/Log | 650 | 1.0 |
| Vybrant DyeCycle | Violet 1 | Peak/Area | 455 | 1.0 |
| Violet | | | | |

### 2.2.6 Data Processing and Analysis

All well-specific flow cytometry data and matching plate map files are transferred to an EC2 server instance on Amazon Web Services (AWS). An automated algorithm converts the raw data to risk scores for each compound in two stages:

### 2.2.6.1 Feature Reduction

For each test compound, ungated FC detection parameters are converted to a feature vector as follows. For each concentration step in a test compound dilution series, quadratic form (QF) distance is calculated between the empirical distribution of a flow cytometry parameter and that same parameter in the negative-control. All QF distance values for the dilution series then form a dose-response distance curve for that FC parameter. The same process is executed for all FC parameters, after which each of these curves is further reduced to two values: the point of the maximum rate of change and the range within which change occurs. By analogy, if a sigmoid curve approximated the observed response, the point of the maximum rate of change would be its inflection point, and the range would be described by the distance between the low and high “plateaus” of the curve. These two values for each FC parameter, point of maximum change and range, are then assembled into a feature vector representing all FC parameters. This vector serves as the quantitative phenotype for the test compound, to be used in subsequent ML classification.

### 2.2.6.2 Machine Learning Classification

Risk scores are produced for test compounds with an ML classifier employing supervised learning with a multidimensional logistic model. The classifier is trained on a set of 300 known compounds drawn from on-market pharmaceuticals, withdrawn drugs, research compounds, and a few industrial/agricultural compounds. First, all training set compounds are assigned to one of two binary classes: the “yes” (expectation of high cell stress) or “no” class. This assignment is based upon manually curated external information from the scientific literature, clinical trial results, and/or known commercial histories. Each training set compound was also screened to produce an empirical phenotypic feature vector, as described above. The classifier is trained by repeated cross-validation. For the two training classes, established from external information, the logistic model optimization process seeks the most parsimonious model allowing for maximum separation of the two populations of phenotypes. The optimally fit model then becomes the classification tool allowing calculation of the probability that a feature vector, from any compound, could be assigned to the “yes” (high cell stress) class. Subsequently, for any test compound, the final multiparametric risk score, or Cell Health Index (CHI), is the probability with which the test compound's phenotypic feature vector can be assigned to the “yes” class defined by the training set. In addition, a series of unidimensional classifiers are trained and applied to the detection parameters separately, calculating the probability of “yes” class assignment if only data for that flow cytometry parameter are considered. These single parameter classifications produce a “fingerprint” of scores that can be interpreted as indicating relative contributions of each parameter to the final multiparameter CHI score. However, note that the predictivity of the individual parameters is not assumed to be equal, among themselves or to the CHI. All test compound results are traceable to specific screen run instances and original compound stocks, regardless of whether any compound name appears more than once within/among screening instances.

### 2.3 Summary of In Vitro Pharmacology Assays

For a detailed description of our collaborators' in vitro pharmacology assay panels, please see the publication by Pottel et al. (Pottel et al., 2020). Briefly, each in vitro assay focuses on one biomolecular target known to be associated with common negative side effects of pharmaceuticals in humans. These targets are generally enzymes, cell surface receptors, or other proteins that mediate cell signal transduction. In one assay panel, chemical compound interaction is assessed for 31 biomolecular targets in a dose-response fashion, which assesses compound-target interaction strength expressed as an IC50 and an activity range (unless no interaction happens). In a second panel, chemical compound interaction is assessed for a further 78 biomolecular targets at one compound concentration only; in this case the assay result is a binary yes/no assessment of target binding at that compound concentration. For each chemical compound that is tested, when results are taken together from all of the in vitro assays, the final assessment of toxicity risk is an interpretation of how “promiscuous” the compound is (how many different biomolecular targets it engaged) and whether or not it potently engaged certain toxicity-associated targets at low concentrations within the dose-response panel of 31 biomolecular targets. This is a somewhat subjective interpretation process; however, as all of the assay targets are known to mediate negative drug side effects, a conservative approach is to treat any chemical compound with caution if it demonstrates strong interaction with even one or a few targets. Alternatively, if there are no strong interactions, but the compound is highly promiscuous as shown by moderate interaction with many of the targets, this may also be an indication that caution is advised during any further development of the compound as a pharmaceutical or excipient.

### 3. RESULTS: COMPARISON OF CELL HEALTH INDEX VALUES WITH IN VITRO PHARMACOLOGY PANELS

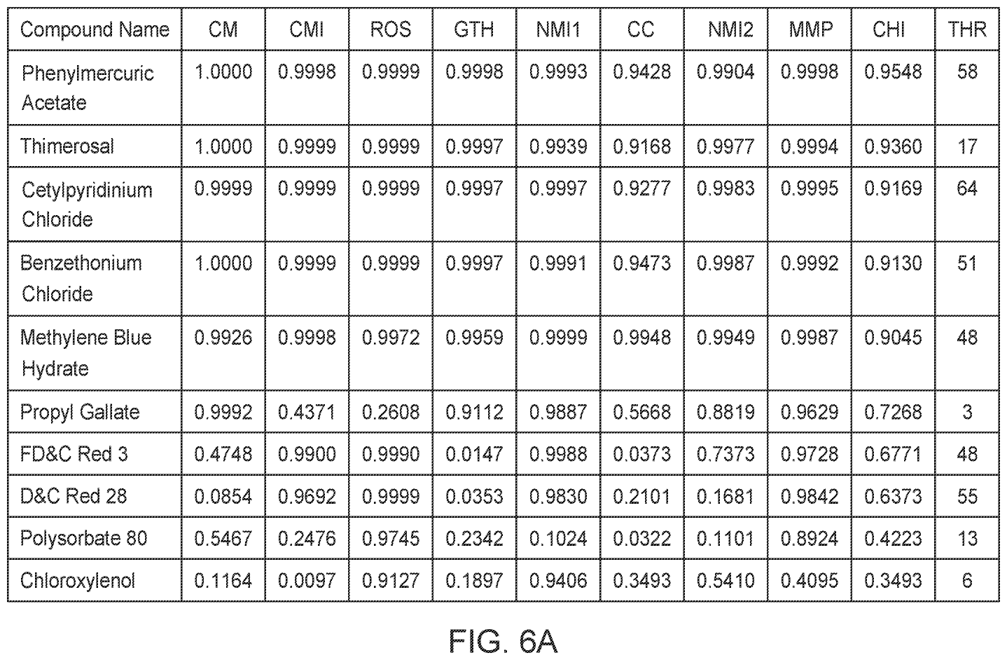

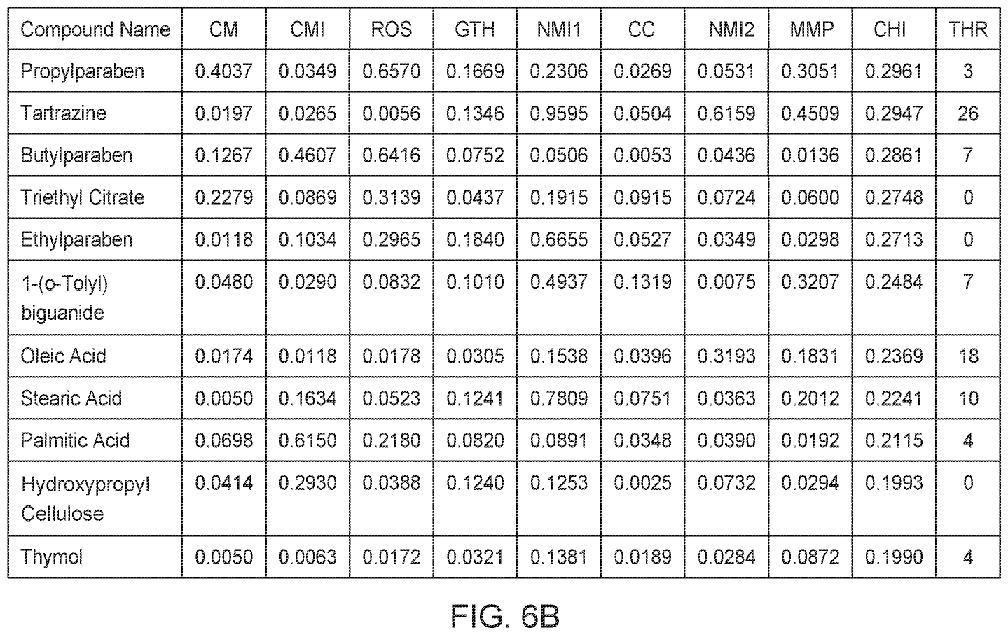

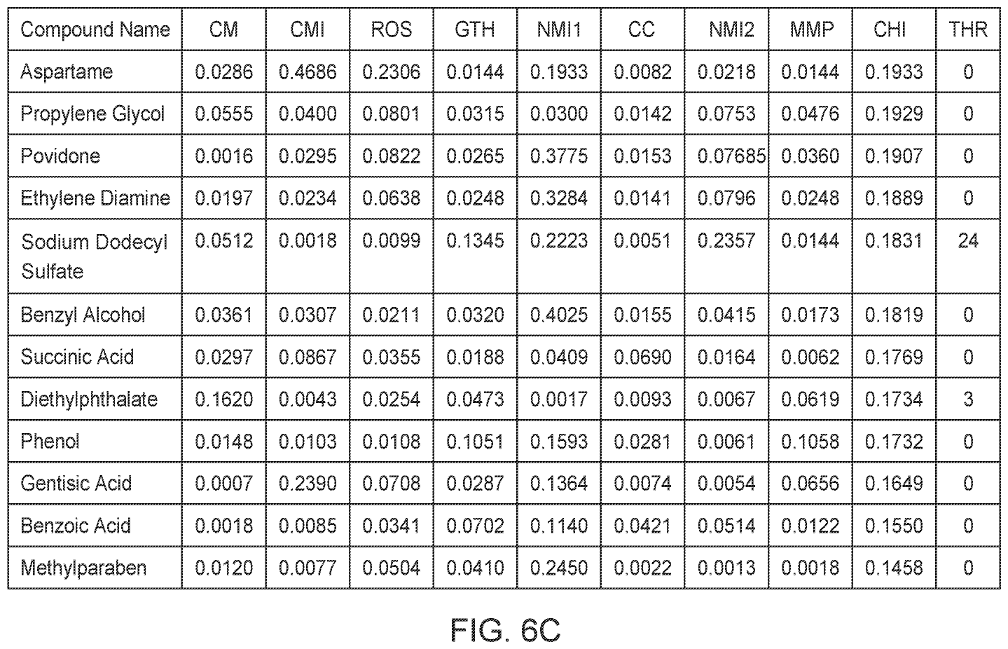

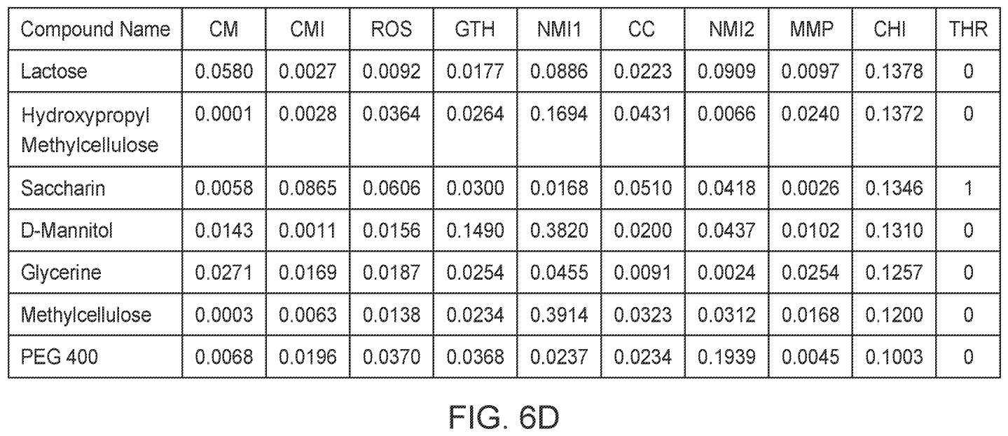

Here we present two summarized versions of the study results. First, for all 40 excipients, FIG. 6 displays ML classifier scores from the Cell Health Screen, including the final Cell Health Index (CHI) and classifier scores for individual biological endpoints, derived by applying subsets of the FC parameters to the classifier. For the biological endpoints, the abbreviation key is as follows: CM=cell morphology, CMI=cell membrane integrity, ROS=reactive oxygen species, GTH=glutathione, NMI1=nuclear membrane integrity 1, CC=cell cycle, NMI2=nuclear membrane integrity 2, MMP=mitochondrial membrane potential. The final column in FIG. 6, “THR”, displays the target hit rate across all of the in vitro pharmacology assays. This is the percentage of all biomolecular targets for which an effect was observed, for each excipient. The THR value serves as an expression of an excipient's promiscuity with regard to binding biomolecular targets known to associate with toxic side effects in humans. FIG. 6 illustrates a distinct, positive association between CHI and THR values. This demonstrates that the Cell Health Screen produces a single probability value, which estimates relative risk of human toxicity, that is generally supported by a chemical compound's degree of interaction with biomolecular targets known to associate with undesired drug side effects.

Second, Table 4 displays results for the excipients with the 11 highest Cell Health Index scores, with a more detailed version of their results from the in vitro pharmacology assay panels. The two most important features to observe are the activity range and average potency, relative to each excipient's CHI score. As CHI begins to substantially decrease for the last three excipients (polysorbate 80, chloroxylenol, and propylparaben), note that there is both a coordinated increase in the low end of the activity range (higher concentration of excipient required to trigger minimal activity) and a coordinated decrease in potency (higher average concentration observed for the IC50 values from dose-response results). These last two features are derived from the first panel of 31 biomolecular targets, which are assayed using a concentration series of each excipient to produce a dose-response curve.

TABLE 4
Target hit rates of selected profiled excipients
in the secondary pharmacology panels.

| | Overall target | Activity | Avg | |
|---|---|---|---|---|
| | hit rate | range | potency | CHI |
| Excipient | (%) | (μM) | (μM) | score |
| Phenylmercuric acetate | 58 | 0.02-22 | 5 | 0.955 |
| Thimerosal | 17 | 0.32-30 | 15 | 0.936 |
| Cetylpyridinium chloride | 64 | 0.55-18 | 9.5 | 0.917 |
| Benzethonium chloride | 51 | 1.1-27 | 7.3 | 0.913 |
| Methylene blue hydrate | 48 | 0.04-24 | 6.5 | 0.905 |
| Propyl gallate | 3 | 0.015-7.2 | 7.2 | 0.727 |
| FD&C Red #3 | 48 | 0.09-25 | 5.7 | 0.677 |
| D&C Red #28 | 55 | 0.13-28 | 10 | 0.637 |
| Polysorbate 80 | 13 | 1.0-21 | 10.7 | 0.422 |
| Chloroxylenol | 6 | 10-14 | 12 | 0.349 |
| Propylparaben | 3 | 11-24 | 19.3 | 0.296 |

This study indicates that the AsedaSciences SYSTEMETRIC Cell Health Screen can serve as an efficient form of triage for eliminating candidate chemical compounds from drug development programs for reasons of toxicity risk. While in vitro pharmacology assay panels can produce useful information related to the same optimization problem, the Cell Health Screen is relatively less labor intensive, less costly, and reduces multidimensional data to single quantitative values requiring no subjective interpretation. As such, the embodiment described above has been reduced to practice in a form with potential to improve state of the art in pharmaceutical development and, possibly, other sectors of the chemical industry.

### 4. REFERENCES

Bora, P., Das, P., Bhattacharyya, R., & Barooah, M. S. (2019). Biocolour: The natural way of colouring food. Journal of Pharmacognosy and Phytochemistry, 8 (3), 3663-3668.
Bowes, J., Brown, A. J., Hamon, J., Jarolimek, W., Sridhar, A., Waldron, G., & Whitebread, S. (2012). Reducing safety-related drug attrition: The use of in vitro pharmacological profiling. Nature Reviews. Drug Discovery, 11 (12), 909-922. https://doi.org/10.1038/nrd3845
Burbacher, T. M., Shen, D. D., Liberato, N., Grant, K. S., Cernichiari, E., & Clarkson, T. (2005). Comparison of blood and brain mercury levels in infant monkeys exposed to methylmercury or vaccines containing thimerosal. Environmental Health Perspectives, 113 (8), 1015-1021. https://doi.org/10.1289/ehp.7712
Chevalier, M., Sakarovitch, C., Precheur, I., Lamure, J., & Pouyssegur-Rougier, V. (2015). Antiseptic mouthwashes could worsen xerostomia in patients taking polypharmacy. Acta Odontologica Scandinavica, 73 (4), 267-273. https://doi.org/10.3109/00016357.2014.923108
Ivanovska, V., Rademaker, C. M. A., van Dijk, L., & Mantel-Teeuwisse, A. K. (2014). Pediatric drug formulations: A review of challenges and progress. Pediatrics, 134 (2), 361-372. https://doi.org/10.1542/peds.2013-3225
Pifferi, G., & Restani, P. (2003). The safety of pharmaceutical excipients. Farmaco (Societa Chimica Italiana: 1989), 58 (8), 541-550. https://doi.org/10.1016/S0014-827X (03) 00079-X
Pottel, J., Armstrong, D., Zou, L., Fekete, A., Huang, X.-P., Torosyan, H., Bednarczyk, D., Whitebread, S., Bhhatarai, B., Liang, G., Jin, H., Ghaemi, S. N., Slocum, S., Lukacs, K. V., Irwin, J. J., Berg, E. L., Giacomini, K. M., Roth, B. L., Shoichet, B. K., & Urban, L. (2020). The activities of drug inactive ingredients on biological targets. Science (New York, N.Y.), 369 (6502), 403-413. https://doi.org/10.1126/science.aaz9906
Rowe, K. S., & Rowe, K. J. (1994). Synthetic food coloring and behavior: A dose response effect in a double-blind, placebo-controlled, repeated-measures study. The Journal of Pediatrics, 125 (5 Pt 1), 691-698. https://doi.org/10.1016/s0022-3476 (94) 70059-1
Walsh, J., Griffin, B. T., Clarke, G., & Hyland, N. P. (2018). Drug-gut microbiota interactions: Implications for neuropharmacology. British Journal of Pharmacology, 175 (24), 4415-4429. https://doi.org/10.1111/bph.14366
Whitebread, S., Hamon, J., Bojanic, D., & Urban, L. (2005). Keynote review: In vitro safety pharmacology profiling: an essential tool for successful drug development. Drug Discovery Today, 10 (21), 1421-1433. https://doi.org/10.1016/S1359-6446 (05) 03632-9
Yang, C., Lim, W., Bazer, F. W., & Song, G. (2018). Butyl paraben promotes apoptosis in human trophoblast cells through increased oxidative stress-induced endoplasmic reticulum stress. Environmental Toxicology, 33 (4), 436-445. https://doi.org/10.1002/tox.22529

All documents referred to in this application by citation are incorporated herein by reference in their entirety, in particular in all parts pertinent to the subject matter for which they have been cited.

1. A cell cytometry method for characterizing the effect of an agent on cells comprising:
contacting aliquots of a population of cells with K different control conditions κ, where K is at least 1, and with I different concentrations i of an agent, where I is at least 1;
measuring P different phenotypic parameters, ψ, in individual cells of each aliquot, where P is at least 2, and where ψₚ denotes a particular phenotypic parameter, thereby obtaining distributions C_{κ} of the measured values for each control condition κ for each phenotypic parameter ψₚ, and distributions Sᵢ of the measured values for each concentration condition i for each phenotypic parameter ψₚ, wherein the phenotypic parameters are measured in the individual cells by cell cytometry using a cell cytometer,
generating, for each concentration i of the agent, a response curve feature vector based on the measurements and indicative of the response of the cells to the agent by:
calculating pairwise distances d between the distributions of measured values at each control condition C_{κ} and each concentration condition Sᵢ separately for each phenotypic parameter ψ, where
$${d}_{κ , i}^{( {ψ}_{p} )} = D ( {C}_{κ}^{( {ψ}_{p} )} , {S}_{i}^{( {ψ}_{p} )} ) , κ = 1 … K , i = 1 … I , p = 2 … P$$
and D is a distance function;
arranging the collected measurements into a tensor A∈ [symbol]
$$A = [ \begin{matrix}{d}_{1 , 1}^{( {ψ}_{1} )} & {d}_{1 , 1}^{( {ψ}_{2} )} & … & {d}_{1 , 1}^{( {ψ}_{p} )} & {d}_{2 , 1}^{( {ψ}_{1} )} & {d}_{2 , 1}^{( {ψ}_{2} )} & … & {d}_{2 , 1}^{( {ψ}_{p} )} & & {d}_{K , 1}^{( {ψ}_{1} )} & {d}_{K , 1}^{( {ψ}_{2} )} & … & {d}_{K , 1}^{( {ψ}_{p} )} \\ {d}_{1 , 2}^{( {ψ}_{1} )} & {d}_{1 , 2}^{( {ψ}_{2} )} & … & {d}_{1 , 2}^{( {ψ}_{p} )} , & {d}_{2 , 2}^{( {ψ}_{1} )} & {d}_{2 , 2}^{( {ψ}_{2} )} & … & {d}_{2 , 2}^{( {ψ}_{p} )} & , … , & {d}_{K , 2}^{( {ψ}_{1} )} & {d}_{K , 2}^{( {ψ}_{2} )} & … & {d}_{K , 2}^{( {ψ}_{p} )} \\ ⋮ & ⋮ & ⋱ & ⋮ & ⋮ & ⋮ & ⋱ & ⋮ & & ⋮ & ⋮ & ⋱ & ⋮ \\ {d}_{1 , I}^{( {ψ}_{1} )} & {d}_{1 , I}^{( {ψ}_{2} )} & … & {d}_{1 , I}^{( {ψ}_{p} )} & {d}_{2 , I}^{( {ψ}_{1} )} & {d}_{2 , I}^{( {ψ}_{2} )} & … & {d}_{2 , I}^{( {ψ}_{p} )} & & {d}_{K , I}^{( {ψ}_{1} )} & {d}_{K , I}^{( {ψ}_{2} )} & … & {d}_{K , I}^{( {ψ}_{p} )}\end{matrix} ] ,$$
calculating for each fiber a_{[κ,ψ]} of the tensor A, a range α between values of distances computed for i=1 and i=I and a maximum rate of change β between values of distances computed for i and i+1, where i takes values from 1 to I−1:
$${α}_{κ}^{( ψ )} = range ( {d}_{κ , 1}^{( ψ )} , {d}_{κ , 2}^{( ψ )} , … , {d}_{κ , I}^{( ψ )} ) , κ = 1 … K \\ {β}_{κ}^{( ψ )} = {max arg}_{i} ( \frac{{d}_{κ , i}^{( ψ )} - {d}_{κ , i + 1}^{( ψ )}}{g ( {S}_{i + 1} ) - g ( {S}_{i} )} ) , κ = 1 … K , i = 1 … I - 1.$$
where g(.) is a transformation function such as generalized logarithm.
Combining, the calculated range α and maximum rate of change β to produce a response curve feature tensor R:
$$R = [ \begin{matrix}{α}_{1}^{( {ψ}_{1} )} & {α}_{1}^{( {ψ}_{2} )} & … & {α}_{1}^{( {ψ}_{p} )} , & {α}_{2}^{( {ψ}_{1} )} & {α}_{2}^{( {ψ}_{2} )} & … & {α}_{2}^{( {ψ}_{p} )} & , … , & {α}_{K}^{( {ψ}_{1} )} & {α}_{K}^{( {ψ}_{2} )} & … & {α}_{K}^{( {ψ}_{p} )} \\ {β}_{1}^{( {ψ}_{1} )} & {β}_{1}^{( {ψ}_{2} )} & … & {β}_{1}^{( {ψ}_{p} )} & {β}_{2}^{( {ψ}_{1} )} & {β}_{2}^{( {ψ}_{2} )} & … & {β}_{2}^{( {ψ}_{p} )} & & {β}_{K}^{( {ψ}_{1} )} & {β}_{K}^{( {ψ}_{2} )} & … & {β}_{K}^{( {ψ}_{p} )}\end{matrix} ]$$
Vectorizing the tensor R to produce curve feature vector r:
$$r = {[ \begin{matrix}{α}_{1}^{( {ψ}_{1} )} , & {α}_{1}^{( {ψ}_{2} )} , & … & {α}_{1}^{( {ψ}_{p} )} , & {β}_{1}^{( {ψ}_{1} )} , & {β}_{1}^{( {ψ}_{2} )} , & … & {β}_{1}^{( {ψ}_{p} )} , \\ {α}_{2}^{( {ψ}_{1} )} , & {α}_{2}^{( {ψ}_{2} )} , & … & {α}_{2}^{( {ψ}_{p} )} , & {β}_{2}^{( {ψ}_{1} )} , & {β}_{1}^{( {ψ}_{2} )} , & … & {β}_{2}^{( {ψ}_{p} )} , \\ {α}_{κ}^{( {ψ}_{1} )} , & {α}_{κ}^{( {ψ}_{2} )} , & … & {α}_{κ}^{( {ψ}_{p} )} , & {β}_{κ}^{( {ψ}_{1} )} , & {β}_{κ}^{( {ψ}_{2} )} , & … & {β}_{κ}^{( {ψ}_{p} )}\end{matrix} ]}^{T}$$
executing a classification model for one or more properties of interest on the generated response curve feature vector r to obtain a likelihood that the agent possesses one or more of said properties.

2. A method according to claim 1, wherein the property is cell toxicity.

3. A method according to claim 1, wherein the property is in vivo toxicity.

4. A method according to claim 1, wherein the phenotypic parameters include any two or more of cell viability, cell cycle stage, mitochondrial membrane integrity, mitochondrial toxicity, glutathione concentration, reactive oxygen species, reducing species, cytoplasmic membrane permeability, DNA damage, a stress response marker, an inflammatory response marker, an apotosis marker and a lipid peroxidase.

5. A method according to claim 1, wherein the phenotypic parameters include any one or more of NFκB, caspase, ERK, SAPK, P13K, AKT, a Bcl-1 family protein, p38, ATM GSk3B and ribosomal S6 kinase.

6. A method according to claim 1, wherein one of the phenotypic parameters is cell cycle state.

7. A method according to claim 1, wherein each population of cells is functionally labeled with a plurality of fluorescence dyes and the phenotypic parameters are detected and quantitated in terms of spectral emission signal(s) that are generated when said populations of labeled cells are subjected to cytometric analysis.

8. The method according to claim 6, wherein a phenotypic parameter is cell cycle and it is quantitated in terms of any one or more of the HOECHST 33342, DRAQ5, YO-PRO-1 IODIDE, DAPI, CYTRAK ORANGE, cyclin or phosphorylated histone protein.

9. A method according to claim 1, wherein the pairwise differences d are normalized to the pairwise difference between a “negative” control and a “positive” control.

10. A method according to claim 1, wherein the differences are calculated by a Wasserstein distance, a quadratic-form distance, a Kolmogorov distance, Sinkhorn distance, or a symmetrized Kullback-Leibler divergence dissimilarity measure.

11. A method according to claim 1, wherein the classification model is a multiple regression model.

12. A method according to claim 1, wherein the classification model is regularized by an elastic net penalty, ridge penalty. LASSO penalty

13. A method according to claim 1, wherein the classification model is trained on response curve feature vectors generated using flow cytometry measurements for cells dosed with known compounds.

14. A system configured to perform a method according to claim 1, comprising in one or more instrumentalities,
a device for carrying out cytometric assays for analysis by flow cytometry;
a flow cytometer configured to carry out multiparametric cytometric assays;
a first computational resource for acquiring and the results of said cytometric assays for further analysis;
a second computational resource for calculating said for each test agent a curve feature vector curve feature vector r:
$$r = {[ \begin{matrix}{α}_{1}^{( {ψ}_{1} )} , & {α}_{1}^{( {ψ}_{2} )} , & … & {α}_{1}^{( {ψ}_{p} )} , & {β}_{1}^{( {ψ}_{1} )} , & {β}_{1}^{( {ψ}_{2} )} , & … & {β}_{1}^{( {ψ}_{p} )} , \\ {α}_{2}^{( {ψ}_{1} )} , & {α}_{2}^{( {ψ}_{2} )} , & … & {α}_{2}^{( {ψ}_{p} )} , & {β}_{2}^{( {ψ}_{1} )} , & {β}_{1}^{( {ψ}_{2} )} , & … & {β}_{2}^{( {ψ}_{p} )} , \\ {α}_{κ}^{( {ψ}_{1} )} , & {α}_{κ}^{( {ψ}_{2} )} , & … & {α}_{κ}^{( {ψ}_{p} )} , & {β}_{κ}^{( {ψ}_{1} )} , & {β}_{κ}^{( {ψ}_{2} )} , & … & {β}_{κ}^{( {ψ}_{p} )}\end{matrix} ]}^{T}$$
and a third computational resource for executing a classification model for one or more properties of interest on said response curve feature vectors r to obtain a likelihood that the agent possesses one or of said properties,
wherein said computational resources may be the same or different computational resources.

15. A method for drug development comprising, for each of a plurality of drug agent candidates:
contacting aliquots of a population of cells with K different control conditions, where K is at least 1, and with/different concentrations i of the agents, where/is at least 1;
measuring P different phenotypic parameters ψ, in individual cells of each aliquot, where P is at least 2, thereby obtaining distributions C_{κ} of the measured values for each control condition κ for each phenotypic parameter ψₚ and distributions Sᵢ of the measured values for each concentration condition i for each phenotypic parameter ψₚ, wherein the phenotypic parameters are measured in the individual cells by cell cytometry using a cell cytometer,
generating, for each concentration i of the agent, a response curve feature vector based on the measurements and indicative of the response of the cells to the agent by:
calculating pairwise distances d between the distributions of each control condition C_{κ} and each concentration condition Sᵢ separately for each phenotypic parameter ψ, where
$${d}_{κ , i}^{( {ψ}_{ρ} )} = D ( {C}_{κ}^{( {ψ}_{ρ} )} , {S}_{j}^{( {ψ}_{ρ} )} ) , κ = 1 … K , i = 1 … I , p = 2 … P$$
and D is a distance function;
arranging the collected measurements into a tensor A∈ [symbol]
$$A = [ \begin{matrix}{d}_{1 , 1}^{( {ψ}_{1} )} & {d}_{1 , 1}^{( {ψ}_{2} )} & … & {d}_{1 , 1}^{( {ψ}_{p} )} & {d}_{2 , 1}^{( {ψ}_{1} )} & {d}_{2 , 1}^{( {ψ}_{2} )} & … & {d}_{2 , 1}^{( {ψ}_{p} )} & & {d}_{K , 1}^{( {ψ}_{1} )} & {d}_{K , 1}^{( {ψ}_{2} )} & … & {d}_{K , 1}^{( {ψ}_{p} )} \\ {d}_{1 , 2}^{( {ψ}_{1} )} & {d}_{1 , 2}^{( {ψ}_{2} )} & … & {d}_{1 , 2}^{( {ψ}_{p} )} , & {d}_{2 , 2}^{( {ψ}_{1} )} & {d}_{2 , 2}^{( {ψ}_{2} )} & … & {d}_{2 , 2}^{( {ψ}_{p} )} & , … , & {d}_{K , 2}^{( {ψ}_{1} )} & {d}_{K , 2}^{( {ψ}_{2} )} & … & {d}_{K , 2}^{( {ψ}_{p} )} \\ ⋮ & ⋮ & ⋱ & ⋮ & ⋮ & ⋮ & ⋱ & ⋮ & & ⋮ & ⋮ & ⋱ & ⋮ \\ {d}_{1 , I}^{( {ψ}_{1} )} & {d}_{1 , I}^{( {ψ}_{2} )} & … & {d}_{1 , I}^{( {ψ}_{p} )} & {d}_{2 , I}^{( {ψ}_{1} )} & {d}_{2 , I}^{( {ψ}_{2} )} & … & {d}_{2 , I}^{( {ψ}_{p} )} & & {d}_{K , I}^{( {ψ}_{1} )} & {d}_{K , I}^{( {ψ}_{2} )} & … & {d}_{K , I}^{( {ψ}_{p} )}\end{matrix} ] ,$$
calculating for each fiber a_{[κ,ψ]} of the tensor A, a range α between values of distances computed for i=1 and i=I and a maximum rate of change β between values of distances computed for every i and i+1, where i takes values from 1 to I−1:
$${α}_{κ}^{( ψ )} = range ( {d}_{κ , 1}^{( ψ )} , {d}_{κ , 2}^{( ψ )} , … , {d}_{κ , I}^{( ψ )} ) , κ = 1 … K \\ {β}_{κ}^{( ψ )} = {max arg}_{i} ( \frac{{d}_{κ , i}^{( ψ )} - {d}_{κ , i + 1}^{( ψ )}}{g ( {S}_{i + 1} ) - g ( {S}_{i} )} ) , κ = 1 … K , i = 1 … I - 1$$
where g(.) is a transformation function such as generalized logarithm.
combining, the calculated range α a maximum rate of change β and to produce a response curve feature tensor R:
$$R = [ \begin{matrix}{α}_{1}^{( {ψ}_{1} )} & {α}_{1}^{( {ψ}_{2} )} & … & {α}_{1}^{( {ψ}_{p} )} , & {α}_{2}^{( {ψ}_{1} )} & {α}_{2}^{( {ψ}_{2} )} & … & {α}_{2}^{( {ψ}_{p} )} & , … , & {α}_{K}^{( {ψ}_{1} )} & {α}_{K}^{( {ψ}_{2} )} & … & {α}_{K}^{( {ψ}_{p} )} \\ {β}_{1}^{( {ψ}_{1} )} & {β}_{1}^{( {ψ}_{2} )} & … & {β}_{1}^{( {ψ}_{p} )} & {β}_{2}^{( {ψ}_{1} )} & {β}_{2}^{( {ψ}_{2} )} & … & {β}_{2}^{( {ψ}_{p} )} & & {β}_{K}^{( {ψ}_{1} )} & {β}_{K}^{( {ψ}_{2} )} & … & {β}_{K}^{( {ψ}_{p} )}\end{matrix} ]$$
Vectorizing the tensor R to produce curve feature vector r:
$$r = {[ \begin{matrix}{α}_{1}^{( {ψ}_{1} )} , & {α}_{1}^{( {ψ}_{2} )} , & … & {α}_{1}^{( {ψ}_{p} )} , & {β}_{1}^{( {ψ}_{1} )} , & {β}_{1}^{( {ψ}_{2} )} , & … & {β}_{1}^{( {ψ}_{p} )} , \\ {α}_{2}^{( {ψ}_{1} )} , & {α}_{2}^{( {ψ}_{2} )} , & … & {α}_{2}^{( {ψ}_{p} )} , & {β}_{2}^{( {ψ}_{1} )} , & {β}_{1}^{( {ψ}_{2} )} , & … & {β}_{2}^{( {ψ}_{p} )} , \\ {α}_{κ}^{( {ψ}_{1} )} , & {α}_{κ}^{( {ψ}_{2} )} , & … & {α}_{κ}^{( {ψ}_{p} )} , & {β}_{κ}^{( {ψ}_{1} )} , & {β}_{κ}^{( {ψ}_{2} )} , & … & {β}_{κ}^{( {ψ}_{p} )}\end{matrix} ]}^{T}$$
executing a classification model for one or more properties of interest on the generated response curve feature vector r to obtain a likelihood that the agent possesses one or more of said properties,
ranking said candidates by the likelihood that they possess said one or more properties,
subjecting each candidate for which said likelihood is above a threshold value to further experimentation and development.

16. A flow cytometer system configured to carry out a carry out a method in accordance with claim 1.
